(19) United States
(12) Patent Application Publication
(10) Pub. No.: US 2005/0277612 A1
(43) Pub. Date: Dec. 15, 2005

(54) Methods, compositions and compound assays for inhibiting amyloid-beta protein production

(76) Inventors: Pascal Gerard Merchiers, Tielen (BE); Koenraad Frederik Florentina Spittaels, Puurs (BE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 11/110,011

(22) Filed: Apr. 20, 2005

Related U.S. Application Data
(60) Provisional application No. 60/563,764, filed on Apr. 20, 2004.

(51) Int. Cl. A61K 48/00; C12Q 1/68; G01N 33/53

(52) U.S. Cl. 514/44; 435/6; 435/7.1

(57) ABSTRACT

A method for identifying compounds that inhibit amyloid-beta precursor protein processing in cells, comprising contacting a test compound with a SPHK polypeptide, or fragment thereof, and measuring a compound-SPHK property related to the production of amyloid-beta peptide. Cellular assays of the method measure indicators including phosphorylated kinase substrate and/or amyloid beta peptide levels. Therapeutic methods, and pharmaceutical compositions including effective amyloid-beta precursor processing-inhibiting amounts of SPHK expression inhibitors, are useful for treating conditions involving cognitive impairment such as Alzheimer's Disease.

### CROSS-REFERENCE TO RELATED APPLICATIONS

This application claims priority to U.S. Provisional Application No. 60/563,764, filed Apr. 20, 2004, the disclosure of which is incorporated herein by reference.

### FIELD OF THE INVENTION

This invention relates to the field of mammalian neuronal cell disorders, and in particular, to methods for identifying effective compounds, and therapies and compositions using such compounds, useful for the prevention and treatment of diseases associated with progressive loss of intellectual capacities in humans.

The neurological disorder that is most widely known for its progressive loss of intellectual capacities is Alzheimer's disease (AD). Worldwide, about 20 million people suffer from Alzheimer's disease. AD is clinically characterized by the initial loss of memory, followed by disorientation, impairment of judgment and reasoning, which is commonly referred to as cognitive impairment, and ultimately by full dementia. AD patients finally lapse into a severely debilitated, immobile state between four and twelve years after onset of the disease.

The key pathological evidence for AD is the presence of extracellular amyloid plaques and intracellular tau tangles in the brain, which are associated with neuronal degeneration (Ritchie and Lovestone (2002)). The extracellular amyloid plaques are believed to result from an increase in the insoluble amyloid beta peptide 1-42 produced by the metabolism of amyloid-beta precursor protein (APP). Following secretion, these amyloid beta 1-42 peptides form amyloid fibrils more readily than the amyloid beta 1-40 peptides, which are predominantly produced in healthy people. It appears that the amyloid beta peptide is on top of the neurotoxic cascade: experiments show that amyloid beta fibrils, when injected into the brains of P301 L tau transgenic mice, enhance the formation of neurofibrillary tangles (Gotz et al. (2001)). In fact, a variety of amyloid beta peptides have been identified as amyloid beta peptides 1-42, 1-40, 1-39, 1-38, 1-37, which can be found in plaques and are often seen in cerebral spinal fluid.

The amyloid beta peptides are generated (or processed) from the membrane anchored APP, after cleavage by beta secretase and gamma secretase at position 1 and 40 or 42, respectively (FIG. 1A)(Annaert and De Strooper (2002)). In addition, high activity of beta secretase results in a shift of the cleavage at position 1 to position 11. Cleavage of amyloid-beta precursor protein by alpha secretase activity at position 17 and gamma secretase activity at 40 or 42 generates the non-pathological p3 peptide. Beta secretase was identified as the membrane anchored aspartyl protease BACE, while gamma secretase is a protein complex comprising presenilin 1 (PS1) or presenilin 2 (PS2), nicastrin, Anterior Pharynx Defective 1 (APH1) and Presenilin Enhancer 2 (PEN2). Of these proteins, the presenilins are widely thought to constitute the catalytic activity of the gamma secretase, while the other components play a role in the maturation and localization of the complex. The identity of the alpha secretase is still illustrious, although some results point towards the proteases ADAM 10 and TACE, which could have redundant functions.

A small fraction of AD cases (mostly early onset AD) are caused by autosomal dominant mutations in the genes encoding presenilin 1 and 2 (PS1; PS2) and the amyloid-beta precursor protein (APP), and it has been shown that mutations in APP, PS1 and PS2 alter the metabolism of amyloid-beta precursor protein leading to such increased levels of amyloid beta 1-42 produced in the brain. Although no mutations in PS1, PS2 and amyloid-beta precursor protein have been identified in late onset AD patients, the pathological characteristics are highly similar to the early onset AD patients. These increased levels of amyloid beta peptide could originate progressively with age from disturbed amyloid-beta precursor protein processing (e.g. high cholesterol levels enhance amyloid beta peptide production) or from decreased amyloid beta peptide catabolism. Therefore, it is generally accepted that AD in late onset AD patients is also caused by aberrant increased amyloid peptide levels in the brains. The level of these amyloid beta peptides, and more particularly amyloid-beta peptide 1-42, is increased in Alzheimer patients compared to the levels of these peptides in healthy persons. Thus, reducing the levels of these amyloid beta peptides is likely to be beneficial for patients with cognitive impairment.

### REPORTED DEVELOPMENTS

The major current AD therapies are limited to delaying progressive memory loss by inhibiting the acetylcholinesterase enzyme, which increases acetylcholine neurotransmitter levels, which fall because the cholinergic neurons are the first neurons to degenerate during AD. This therapy does not halt the progression of the disease.

Therapies aimed at decreasing the levels of amyloid beta peptides in the brain, are increasingly being investigated and focus on the perturbed amyloid-beta precursor protein processing involving the beta- or gamma secretase enzymes.

The present invention is based on the discovery that certain known polypeptides are factors in the up-regulation and/or induction of amyloid beta precursor processing in neuronal cells, and that the inhibition of the function of such polypeptides are effective in reducing levels of amyloid beta peptides.

### SUMMARY OF THE INVENTION

The present invention relates to the relationship between the function of sphingosine kinases (“SPHKs”) and amyloid-beta precursor protein processing in mammalian cells.

One aspect of the present invention is a method for identifying a compound that inhibits the processing of amyloid-beta precursor protein in a mammalian cell, comprising
(a) contacting a compound with a polypeptide comprising an amino acid sequence selected from the group consisting of SEQ ID NO: 3 and 4; and
(b) measuring a compound-polypeptide property related to the production of amyloid-beta peptide.

Aspects of the present method include the in vitro assay of compounds using polypeptide of a SPHK, and cellular assays wherein SPHK inhibition is followed by observing indicators of efficacy, including phosphorylated kinase substrate levels and/or amyloid beta peptide levels.

Another aspect of the invention is a method of treatment or prevention of a condition involving cognitive impairment, or a susceptibility to the condition, in a subject suffering or susceptible thereto, by administering a pharmaceutical composition comprising an effective amyloid-beta precursor processing-inhibiting amount of a SPHK inhibitor.

A further aspect of the present invention is a pharmaceutical composition for use in said method wherein said inhibitor comprises a polynucleotide selected from the group of an antisense polynucleotide, a ribozyme, and a small interfering RNA (siRNA), wherein said agent comprises a nucleic acid sequence complementary to, or engineered from, a naturally occurring polynucleotide sequence encoding a polypeptide, comprising an amino acid sequence selected from the group consisting of SEQ ID NO: 3-4, or a fragment thereof, Another further aspect of the present invention is a pharmaceutical composition comprising a therapeutically effective amyloid-beta precursor processing-inhibiting amount of a SPHK inhibitor or its pharmaceutically acceptable salt, hydrate, solvate, or prodrug thereof in admixture with a pharmaceutically acceptable carrier. The present polynucleotides and SPHK inhibitor compounds are also useful for the manufacturing of a medicament for the treatment of Alzheimer's disease.

### BRIEF DESCRIPTION OF THE DRAWINGS

FIG. 1A: APP processing: The membrane anchored amyloid precursor protein (APP) is processed by two pathways: the amyloidogenic and non amyloidogenic pathway. In the latter pathway, APP is cleaved first by alpha secretase and then by gamma secretase, yielding the p3 peptides (17-40 or 17-42). The amyloidogenic pathway generates the pathogenic amyloid beta peptides (A beta) after cleavage by beta- and gamma-secretase respectively. The numbers depicted are the positions of the amino acids comprising the A beta sequences.

FIG. 2: The metabolism and catabolism of sphingosine 1 phosphate (S1P). S1P is generated by the conversion of sphingosine to S1P by sphingosine kinase. S1P can be metabolized by S1P lyase to ethanolamine phosphate and palmitic acid, which are then further metabolized to a variety of lipids and phosphatidylethanolamine (PE). Conversely, S1P phosphatase regenerates sphingosine from S1P.

FIG. 3: Evaluation of the APP processing assay: Positive (PSIG384L; PSIL392V and BACE1) and negative (eGFP, LacZ and empty) control viruses are infected in Hek293APPwt at random MOI, mimicking a screening. A and B: Transduction is performed respectively with 1 and 0.2 μl of virus and amyloid beta 1-42 levels are performed. Data are represented as relative light units and correlate to pM of amyloid beta 1-42.

FIG. 4: Modulation of amyloid beta peptide levels by overexpression of SPHK polypeptides in Hek293 APPwt cells: Hek293 APPwt cells are transduced with increasing MOI of empty adenovirus and adenoviruses harbouring cDNAs expressing the SPHK polypeptides, PS1 G384L, eGFP and LacZ. Amyloid beta peptide levels are monitored through the amyloid beta 1-42, amyloid beta 1-40, amyloid beta 1-x and amyloid beta 11-42 ELISAs.

FIG. 5: Modulation of amyloid beta 1-42 peptide levels by knock down of SPHK in SH-SY5Y APPwt cells: SH-SY5Y APPwt cells are infected with GL2 and SPHK2 KD viruses. In addition, an adenovirus expressing the APP Swedish mutant is co-infected. SPHK2 RNA levels in the infected cells are determined using real time PCR and are normalized against the GAPDH RNA levels (A). Data are represented relative to GAPDH levels. The amyloid beta 1-42 levels in the conditioned medium are determined by ELISA (B). Data are represented as relative light units and correlate to pM of amyloid beta 1-42.

FIG. 6: Amyloid beta peptide fingerprint generated by overexpression of the SPHK polypeptides: Hek293 APPwt cells were infected with an adenovirus harbouring the SPHK2 polynucleotides and the LacZ cDNA. Conditioned medium was collected and 4G8 immuno-precipitated amyloid beta peptides were analysed by mass spectroscopy. Levels of the amyloid beta peptides were determined relative to an exogenously added amyloid beta 12-18 standard peptide.

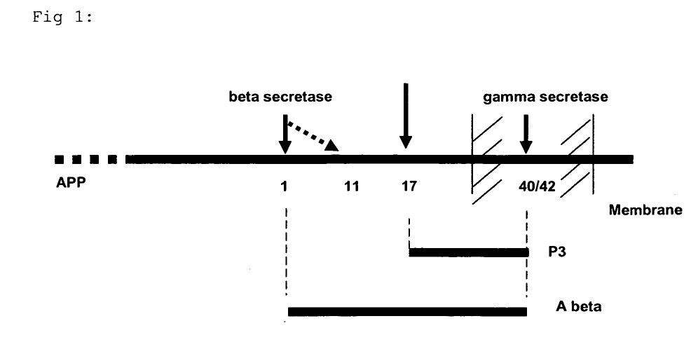

### DETAILED DESCRIPTION

The following terms are intended to have the meanings presented therewith below and are useful in understanding the description of and intended scope of the present invention.

Definitions:

The term “amyloid beta peptide” means amyloid beta peptides processed from the amyloid beta precursor protein (APP). The most common peptides include amyloid beta peptides 1-40, 1-42, 11-40 and 11-42. Other species less prevalent amyloid beta peptides are described as y-42, whereby y ranges from 2-17, and 1-x whereby x ranges from 24-39 and 41.

The term “carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN.TM., polyethylene glycol (PEG), and PLURONICS.TM.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “contact” or “contacting” means bringing at least two moieties together, whether in an in vitro system or an in vivo system.

The term “condition” or “disease” means the overt presentation of symptoms (i.e., illness) or the manifestation of abnormal clinical indicators (e.g., biochemical indicators), resulting from defects in one amyloid beta protein precursor processing. Alternatively, the term “disease” refers to a genetic or environmental risk of or propensity for developing such symptoms or abnormal clinical indicators.

The term “endogenous” shall mean a material that a mammal naturally produces. Endogenous in reference to, for example and not limitation, the term “kinase” shall mean that which is naturally produced by a mammal (for example, and not limitation, a human) or a virus. In contrast, the term non-endogenous in this context shall mean that which is not naturally produced by a mammal (for example, and not limitation, a human) or a virus. Both terms can be utilized to describe both “in vivo” and “in vitro” systems. For example, and not a limitation, in a screening approach, the endogenous or non-endogenous kinase may be in reference to an in vitro screening system. As a further example and not limitation, where the genome of a mammal has been manipulated to include a non-endogenous constitutively activated kinase, screening of a candidate compound by means of an in vivo system is viable.

The term “expression” comprises both endogenous expression and overexpression by transduction.

The term “expressible nucleic acid” means a nucleic acid coding for a proteinaceous molecule, an RNA molecule, or a DNA molecule.

The term “hybridization” means any process by which a strand of nucleic acid binds with a complementary strand through base pairing. The term “hybridization complex” refers to a complex formed between two nucleic acid sequences by virtue of the formation of hydrogen bonds between complementary bases. A hybridization complex may be formed in solution (e.g., Cₒₜ.or Rₒₜ analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed). The term “stringent conditions” refers to conditions that permit hybridization between polynucleotides and the claimed polynucleotides. Stringent conditions can be defined by salt concentration, the concentration of organic solvent, e.g., formamide, temperature, and other conditions well known in the art. In particular, reducing the concentration of salt, increasing the concentration of formamide, or raising the hybridization temperature can increase stringency.

The term “inhibit” or “inhibiting”, in relationship to the term “response” means that a response is decreased or prevented in the presence of a compound as opposed to in the absence of the compound.

The term “ligand” means an endogenous, naturally occurring molecule specific for an endogenous, naturally occurring receptor.

The term “pharmaceutically acceptable prodrugs” as used herein means the prodrugs of the compounds useful in the present invention, which are, within the scope of sound medical judgment, suitable for use in contact with the tissues of patients with undue toxicity, irritation, allergic response commensurate with a reasonable benefit/risk ratio, and effective for their intended use of the compounds of the invention. The term “prodrug” means a compound that is transformed in vivo to yield an effective compound useful in the present invention or a pharmaceutically acceptable salt, hydrate or solvate thereof. The transformation may occur by various mechanisms, such as through hydrolysis in blood. The compounds bearing metabolically cleavable groups have the advantage that they may exhibit improved bioavailability as a result of enhanced solubility and/or rate of absorption conferred upon the parent compound by virtue of the presence of the metabolically cleavable group, thus, such compounds act as pro-drugs. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8, 1-38, (1992); J. Pharm. Sci., 77, 285 (1988); Chem. Pharm. Bull., N. Nakeya et al, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference. An example of the prodrugs is an ester prodrug. “Ester prodrug” means a compound that is convertible in vivo by metabolic means (e.g., by hydrolysis) to an inhibitor compound according to the present invention. For example an ester prodrug of a compound containing a carboxy group may be convertible by hydrolysis in vivo to the corresponding carboxy group.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” means a polynucleic acid, in single or double stranded form, and in the sense or antisense orientation, complementary polynucleic acids that hybridize to a particular polynucleic acid under stringent conditions, and polynucleotides that are homologous in at least about 60 percent of its base pairs, and more preferably 70 percent of its base pairs are in common, most preferably 90 percent, and in a special embodiment 100 percent of its base pairs. The polynucleotides include polyribonucleic acids, polydeoxyribonucleic acids, and synthetic analogues thereof. The polynucleotides are described by sequences that vary in length, that range from about 10 to about 5000 bases, preferably about 100 to about 4000 bases, more preferably about 250 to about 2500 bases. A preferred polynucleotide embodiment comprises from about 10 to about 30 bases in length. A special embodiment of polynucleotide is the polyribonucleotide of from about 10 to about 22 nucleotides, more commonly described as small interfering RNAs (siRNAs). Another special embodiment are nucleic acids with modified backbones such as peptide nucleic acid (PNA), polysiloxane, and 2′-O-(2-methoxy)ethylphosphorothioate, or including non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection.

The term “polypeptide” relates to proteins (such as kinases, proteases, SPHKs), proteinaceous molecules, fractions of proteins peptides and oligopeptides.

The term “solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “effective amount” or “therapeutically effective amount” means that amount of a compound or agent that will elicit the biological or medical response of a subject that is being sought by a medical doctor or other clinician. In particular, with regard to treating an neuronal disorder, the term “effective amount” is intended to mean that effective amyloid-beta precursor processing inhibiting amount of an compound or agent that will bring about a biologically meaningful decrease in the levels of amyloid beta peptide in the subject's brain tissue.

The term “treating” means an intervention performed with the intention of preventing the development or altering the pathology of, and thereby alleviating a disorder, disease or condition, including one or more symptoms of such disorder or condition. Accordingly, “treating” refers to both therapeutic treatment and prophylactic or preventative measures. Those in need of treating include those already with the disorder as well as those in which the disorder is to be prevented. The related term “treatment,” as used herein, refers to the act of treating a disorder, symptom, disease or condition, as the term “treating” is defined above.

The background of the present inventors' discovery is described briefly below.

Background of the Sphingosine Kinases:

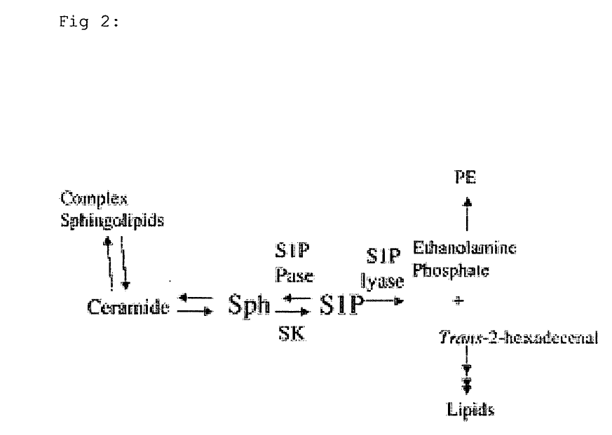

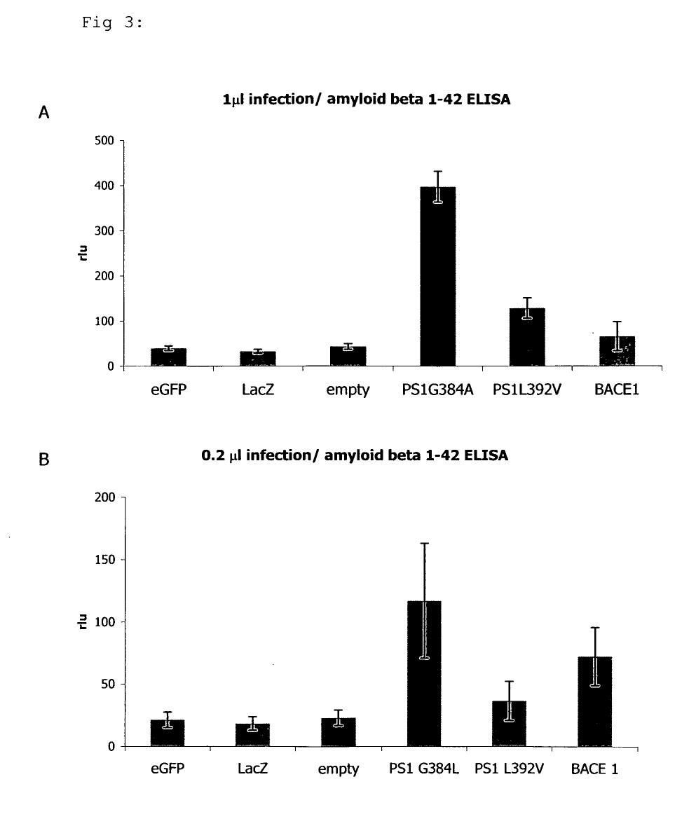

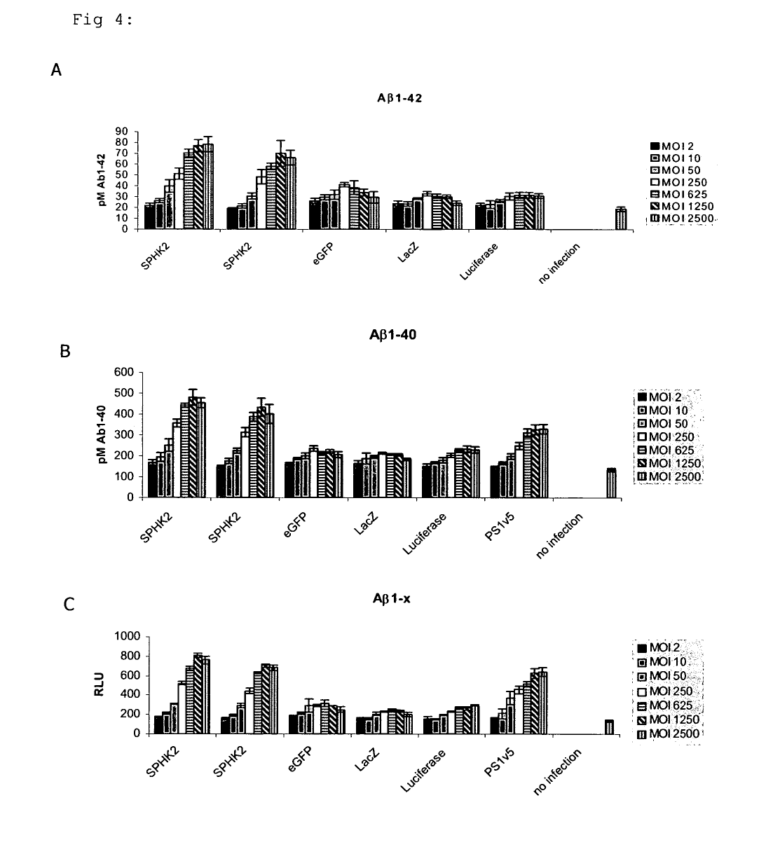

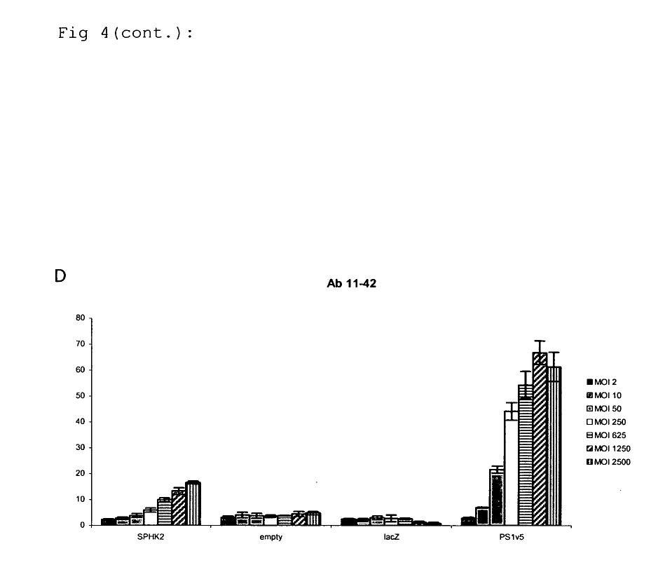

Sphingosine kinases catalyze the phosphorylation of sphingosine that produces sphingosine-1-phosphate (SIP) (see FIG. 2). This bioactive lipid mediator appears to function as both an intracellular second messenger and a ligand of cell-surface receptors, and is implicated in the regulation of cell proliferation and anti-apoptotic processes. Sphingosine kinase is activated upon stimulation of the cells by tumor necrosis factor-alpha, platelet-derived growth factor, nerve growth factor, n-formyl-methionyl-leucyl-phenylalanine, phorbol esters, muscarinic acetylcholine agonists etc. Besides this inducible sphingosine kinase activity, there is considerable basal activity found in un-stimulated cells as well. Two human isoforms of SPHK (hSPHK1 and hSPHK2) have been cloned and characterized (hSPHK2: Liu et al. (2000); hSPHK1: Pitson et al. (2000)). The cDNA sequences for SPHK1 and SPHK2 are identified in Table 1.

TABLE 1
cDNA SEQ IDs:

| | | | SEQ ID NO: | |
|---|---|---|---|---|
| Accession | Description | Code | DNA | Protein |
| NM_020126 | Sphingosine kinase 2 | SPHK2 | 1 | 3 |
| NM_021972 | Sphingosine kinase 1 | SPHK1 | 2 | 4 |

Although highly similar in amino acid sequence, SPHK2 diverges in its amino terminus and central region from SPHK1. From a structural point of view, sphingosine kinases do not contain recognizable catalytic or substrate-binding sites, compared to sequence motifs found in other kinases. They only share some sequence and likely structural similarities with the highly conserved glycine-rich loop, which is known to be involved in anchoring and positioning the nucleotide in the catalytic site of many protein kinases (Pitson et al. (2002)). The sphingosine kinases do, however, have sequence similarity to the putative catalytic domain of diacylglycerol kinases. These structural differences with “classic” kinases may be one of the reasons why sphingosine kinase inhibitors have not been widely studied. Until now, pharmacological studies of SPHKs could only apply the sole existing lipid inhibitors of these kinases. Since accumulating evidence points to the pivotal role of SPHKs in the regulation of tumor growth, the pharmacological and academic world started, though only recently, the search for nonlipid synthetic compound inhibitors (French et al., (2003)).

SPHK1 and SPHK2 catalyse the formation of SIP but do exhibit different kinetic properties for this enzymatic reaction. In addition, they differ in developmental expression (SPHK2 appears later in development than SPHK1), in tissue distribution (SPHK2 is highly expressed in brain, whereas SPHK1 only modestly) and in sub cellular localisation. SPHK1 predominantly localises in the cytosol, whereas SPHK2 mainly localises in the nucleus (depending on cell type and density). These findings suggest that SPHK1 and SPHK2 may have distinct physiological functions. Indeed, partially, SPHK1 and SPHK2 even provoke opposite downstream effects. Upon overexpression, SPHK1 induces cell proliferation by promoting the G1 to S transition of the cell cycle as well as by inhibiting apoptotic signals, and it stimulates DNA synthesis. However, when over-expressed, SPHK2 causes inhibition of DNA synthesis in various cell types. Hence, in contrast to SPHK1, SPHK2 enhances apoptosis and suppresses cellular proliferation. This difference can be ascribed to their specific sub cellular localisation. In fact, SPHK1 artificially directed to the nucleus exhibit the same properties as SPHK2.

Two recent seemingly contradictory studies demonstrate that blocking in a cell the synthesis of ceramide (the precursor of sphingosine) thereby reducing all metabolic products of ceramide, as well as exogenously increasing the presence of one ceramide metabolite, sphingosine, both increase amyloid precursor protein cleavage. Sawamura and colleagues (2004) reported that blockage of ceramide synthesis increased the secretion of amyloid beta 42 increased and did not alter of amyloid beta 40, whereas Puglielli and his group (2003) reported an increase in amyloid beta 42 by increasing levels of ceramide. However, these reports do not suggest or disclose (1) the relationship between SPHKs and amyloid beta production/secretion, and (2) the present discovery that overexpression of SPHK2 increases and knock-down of SPHK2 reduces amyloid beta 1-42 in the conditioned medium of transduced cells.

### REFERENCES

Annaert, W. and B. De Strooper (2002). “A cell biological perspective on Alzheimer's disease.” Annu Rev Cell Dev Biol 18: 25-51.
French, K. J., Schrecengost, R. S., Lee, B. D., Zhuang, Y., Smith, S. N., Eberly, J. L., Yun, J. K. and Smith, C. D. (2003). “Discovery and evaluation of inhibitors of human sphingosine kinase.” Cancer Research. 63: 5962-5969 Gotz, J., F. Chen, et al. (2001). “Formation of neurofibrillary tangles in P3011 tau transgenic mice induced by Abeta 42 fibrils.” Science 293(5534): 1491-5.
Hartmann, T. (2001). “Cholesterol, Abeta and Alzheimer's disease.” Trends in Neurosci. 24: S45-48.
Lipinski, C. A., Lombardo, F., Dominy, B. W., and Feeney, P. J. “Experimental and computational approaches to estimate solubility and permeability in drug discovery and development settings” Adv. Drug. Deliv. Rev., 23, 3-25, 1997.
Liu, H., Sugiura, M., Nava, V. E., Edsall, L. C., Kono, K., Poulton, S., Milstien, S, Kohama, T. and Spiegel, S. (2000). “Molecular cloning and functional characterization of a novel mammalian sphingosine kinase type 2 isoform.” The Journal of Biological Chemistry. 275, 26: 19513-19520.
Pitson, S. M., D'Andrea, R. J., Vandeleur, P. A. B., Xia, P., Gamble, J. R., Vadas, M. A. And Wattenberg, B. W. (2000). “Human sphingosine kinase: purification, molecular cloning and characterization of the native and recombinant enzymes.” Biochem. Journal. 350: 429-441.
Pitson, S. M., Moretti, P. A. B., Zebol, J. R., Zareie, R., Derian, C. K., Darrow, A. L., Qi, J., D'Andrea R. J., Bagley, C. J., Vadas, M. A. and Wattenberg, B. W. (2002). “The nucleotide binding site of human sphingosine kinase 1.” The Journal of Biological Chemistry. 277, 51: 49545-49553.
Puglielli, L., Ellis, B. C., Saunders, A. J. and Kovacs, D. (2003). “Ceramide stabilizes BACE1 and promotes amyloid beta-peptide biogenesis.” The Journal of Biological Chemistry. 278, 22: 19777-19783.
Ritchie, K. and S. Lovestone (2002). “The dementias.” Lancet 360(9347): 1759-66.
Sawamura, N., Ko, M., Yu, W., Zou, K., Hanada, K., Suzuki, T., Gong, J-S., Yanagisawa, K. And Michikawa, M. (2004). “Modulation of amyloid precursor protein cleavage by cellular sphingolipids.” The Journal of Biological Chemistry. Epub.
   Applicants' Invention Based on SPHK Relationship to Amyloid Beta Peptides

As noted above, the present invention is based on the present inventors' discovery that SPHK(s) are factors in the up-regulation and/or induction of amyloid beta precursor processing in mammalian, and principally, neuronal cells, and that the inhibition of the function of such polypeptides is effective in reducing levels of amyloid beta protein peptides.

The present inventors are unaware of any prior knowledge linking SPHKs, and more particularly SPHK1 and SPHK2, and amyloid beta peptide formation and secretion. As discussed in more detail in the Experimental section below, the present inventors demonstrate that the knockdown of SPHK1 and SPHK2 reduces amyloid beta 1-42 in the conditioned medium of transduced cells. The present invention is based on these findings and the recognition that the SPHKs may be putative drug targets for Alzheimer's disease.

One aspect of the present invention is a method based on the aforesaid discovery for identifying a compound that inhibits the processing of amyloid-beta precursor protein in a mammalian cell, and may therefore be useful in reducing amyloid beta peptide levels in a subject. The present method comprises contacting a drug candidate compound with a SPHK polypeptide, or a fragment of said polypeptide, and measuring a compound-polypeptide property related to the production of amyloid-beta protein. The “compound-polypeptide property” is a measurable phenomenon chosen by the person of ordinary skill in the art, and based on the recognition that SPHK activation and deactivation is a causative factor in the activation and deactivation, respectively, of amyloid beta protein precursor processing, and an increase and decrease, respectively, of amyloid beta peptide levels. The measurable property may range from the binding affinity for a peptide domain of the SPHK polypeptide, to the level of any one of a number of phosphorylated kinase substrate levels resulting from the activation or deactivation of the SPHK, to a reporter molecule property directly linked to the aforesaid phosphorylated substrate, and finally to the level of amyloid beta peptide secreted by the mammalian cell contacted with the compound.

Depending on the choice of the skilled artisan, the present assay method may be designed to function as a series of measurements, each of which is designed to determine whether the drug candidate compound is indeed acting on SPHK to thereby facilitate the amyloid beta peptide pathway. For example, an assay designed to determine the binding affinity of a compound to SPHK, or fragment thereof, may be necessary, but not sufficient, to ascertain whether the test compound would be useful for reducing amyloid beta peptide levels when administered to a subject. Nonetheless, such binding information would be useful in identifying a set of test compounds for use in an assay that would measure a different property, further down the biochemical pathway. Such second assay may be designed to confirm that the test compound, having binding affinity for a SPHK peptide, actually down-regulates or inhibits SPHK function in a mammalian cell. This further assay may measure a phosphorylated kinase substrate that is a direct consequence of the activation or deactivation of the SPHK, or a synthetic reporter system responding thereto. Measuring a different phosphorylated kinase substrate, and/or confirming that the assay system itself is not being affected directly in contrast to the SPHK pathway may further validate the assay. In this latter regard, suitable controls should always be in place to insure against false positive readings.

The order of taking these measurements is not believed to be critical to the practice of the present invention, which may be practiced in any order. For example, one may first perform a screening assay of a set of compounds for which no information is known respecting the compounds' binding affinity for SPHK. Alternatively, one may screen a set of compounds identified as having binding affinity for a SPHK peptide domain, or a class of compounds identified as being an inhibitor of a SPHK. However, for the present assay to be meaningful to the ultimate use of the drug candidate compounds, a measurement of the phosphorylated kinase substrate(s), or the ultimate amyloid beta peptide levels, is necessary. Validation studies including controls, and measurements of binding affinity to SPHK are nonetheless useful in identifying a compound useful in any therapeutic or diagnostic application.

The present assay method may be practiced in vitro, using one or more of the SPHK proteins, or fragments thereof. The amino acid sequences of the SPHKs are found in SEQ ID NO: 3 and 4. The binding affinity of the compound with the polypeptide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer. The binding affinity of compounds can also be expressed in dissociation constant (Kd) or as IC50 or EC50. The IC50 represents the concentration of a compound that is required for 50% inhibition of binding of another ligand to the polypeptide. The EC50 represents the concentration required for obtaining 50% of the maximum effect in any assay that measures receptor function. The dissociation constant, Kd, is a measure of how well a ligand binds to the polypeptide, it is equivalent to the ligand concentration required to saturate exactly half of the binding-sites on the polypeptide. Compounds with a high affinity binding have low Kd, IC50 and EC50 values, i.e. in the range of 100 nM to 1 pM; a moderate to low affinity binding relates to a high Kd, IC50 and EC50 values, i.e. in the micromolar range.

The present assay method may also be practiced in a cellular assay, A host cell expressing SPHK can be a cell with endogenous expression or a cell over-expressing the SPHK e.g. by transduction. When the endogenous expression of the polypeptide is not sufficient to determine a baseline that can easily be measured, one may use using host cells that over-express SPHK. Over-expression has the advantage that the level of the phosphorylated kinase substrate is higher than the activity level by endogenous expression. Accordingly, measuring such levels using presently available techniques is easier. In such cellular assay, the biological activity of SPHK may be measured by following the production of a phosphorylated kinase substrate, such as sphingosine-1-phosphate, which is preferred phosphorylated kinase substrate to measure. Phosphorylated kinase substrate levels may be measured by several different techniques, either directly by ELISA or radioactive technologies or indirectly by reporter gene analysis, discussed below. Increased presence of SPHK in a cell increases the level of secreted amyloid beta peptides.

The present invention further relates to a method for identifying a compound that inhibits amyloid-beta precursor protein processing in a mammalian cell comprising:
(a) contacting a compound with a polypeptide comprising an amino acid sequence selected from the group consisting of SEQ ID NO: 3-4,
(b) determining the binding affinity of the compound to the polypeptide,
(c) contacting a population of mammalian cells expressing said polypeptide with the compound that exhibits a binding affinity of at least 10 micromolar, and
(d) identifying the compound that inhibits the amyloid-beta precursor protein processing in the cells.

A further embodiment of the present invention relates a method to identify a compound that inhibits the amyloid-beta precursor protein processing in a cell, wherein the activity level of the SPHK polypeptide is measured by determining the level of one or more phosphorylated kinase substrates, wherein the level of the one or phosphorylated kinase substrate is determined with a reporter controlled by a promoter, which is responsive to the phosphorylated kinase substrate. The reporter is a reporter gene under the regulation of a promoter that responds to the cellular level of phosphorylated kinase substrates. A preferred phosphorylated kinase substrate is sphingosine. The reporter gene should have a gene product that is easily detected, and that may be stably infected in the host cell. Such methods are well known by any person with ordinary skill in the art.

The reporter gene may be selected from alkaline phosphatase, green fluorescent protein (GFP), enhanced green fluorescent protein (eGFP), destabilized green fluorescent protein (dGFP), luciferase, and beta-galactosidase among others. The reporter is preferably luciferase or beta-galactosidase, which are readily available and easy to measure over a large range of activites. The promoter in the reporter construct is preferably a cyclic AMP-responsive promoter, an AP-1 responsive promoter, or a NF-AT responsive promoter. The cyclic-AMP responsive promoter is responsive to the cyclic-AMP levels in the cell. The NF-AT responsive promoter is sensitive to cytoplasmic Ca²⁺-levels in the cell. The AP-1 responsive promoter is sensitive for activated by Mitogen-activated protein kinase (MAPK) pathways in the cell.

A further embodiment of the present invention relates a method to identify a compound that inhibits the amyloid-beta precursor protein processing in a cell, wherein the activity level of the SPHK polypeptide is measured by determining the level of amyloid beta peptides. The levels of these peptides may be measured with specific ELISAs using antibodies specifically recognizing the different amyloid beta peptide species (see e.g. Example 1). Secretion of the various amyloid beta peptides may also be measured using antibodies that bind all peptides. Levels of amyloid beta peptides can also be measured by Mass spectrometry analysis.

For high-throughput purposes, libraries of compounds may be used such as antibody fragment libraries, peptide phage display libraries, peptide libraries (e.g. LOPAP™, Sigma Aldrich), lipid libraries (BioMol), synthetic compound libraries (e.g. LOPAC™, Sigma Aldrich) or natural compound libraries (Specs, TimTec).

Preferred drug candidate compounds are low molecular weight compounds. Low molecular weight compounds, i.e. with a molecular weight of 500 Dalton or less, are likely to have good absorption and permeation in biological systems and are consequently more likely to be successful drug candidates than compounds with a molecular weight above 500 Dalton (Lipinski et al. (1997)). Peptides comprise another preferred class of drug candidate compounds. Peptides may be excellent drug candidates and there are multiple examples of commercially valuable peptides such as fertility hormones and platelet aggregation inhibitors. Natural compounds are another preferred class of drug candidate compound. Such compounds are found in and extracted from natural sources, and which may thereafter be synthesized. The lipids are another preferred class of drug candidate compound.

Another preferred class of drug candidate compounds is an antibody. The present invention also provides antibodies directed against SPHK. These antibodies should be endogenously produced to bind to the intra-cellular SPHK domain. These antibodies may be monoclonal antibodies or polyclonal antibodies. The present invention includes chimeric, single chain, and humanized antibodies, as well as FAb fragments and the products of a FAb expression library, and Fv fragments and the products of an Fv expression library.

In certain embodiments, polyclonal antibodies may be used in the practice of the invention. The skilled artisan knows methods of preparing polyclonal antibodies. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. Antibodies may also be generated against the intact SPHK protein or polypeptide, or against a fragment such as its extracellular domain peptides, derivatives including conjugates, or other epitope of the SPHK protein or polypeptide, such as the SPHK embedded in a cellular membrane, or a library of antibody variable regions, such as a phage display library.

It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants that may be employed include Freund's complete adjuvant and MPL-TDM adjuvant (monophosphoryl Lipid A, synthetic trehalose dicorynomycolate). One skilled in the art without undue experimentation may select the immunization protocol.

In some embodiments, the antibodies may be monoclonal antibodies. Monoclonal antibodies may be prepared using methods known in the art. The monoclonal antibodies of the present invention may be “humanized” to prevent the host from mounting an immune response to the antibodies. A “humanized antibody” is one in which the complementarity determining regions (CDRs) and/or other portions of the light and/or heavy variable domain framework are derived from a non-human immunoglobulin, but the remaining portions of the molecule are derived from one or more human immunoglobulins. Humanized antibodies also include antibodies characterized by a humanized heavy chain associated with a donor or acceptor unmodified light chain or a chimeric light chain, or vice versa. The humanization of antibodies may be accomplished by methods known in the art (see, e.g. Mark and Padlan, (1994) “Chapter 4. Humanization of Monoclonal Antibodies”, The Handbook of Experimental Pharmacology Vol. 113, Springer-Verlag, New York). Transgenic animals may be used to express humanized antibodies.

Human antibodies can also be produced using various techniques known in the art, including phage display libraries (Hoogenboom and Winter, (1991) J. Mol. Biol. 227:381-8; Marks et al. (1991). J. Mol. Biol. 222:581-97). The techniques of Cole, et al. and Boerner, et al. are also available for the preparation of human monoclonal antibodies (Cole, et al. (1985) Monoclonal Antibodies and Cancer Therapy, Alan R. Liss, p. 77; Boerner, et al (1991). J. Immunol., 147(1):86-95).

Techniques known in the art for the production of single chain antibodies can be adapted to produce single chain antibodies to the SPHK polypeptides and proteins of the present invention. The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain cross-linking. Alternatively; the relevant cysteine residues are substituted with another amino acid residue or are deleted so as to prevent cross-linking.

Bispecific antibodies are monoclonal, preferably human or humanized, antibodies that have binding specificities for at least two different antigens and preferably for a cell-surface protein or receptor or receptor subunit. In the present case, one of the binding specificities is for one extracellular domain of the SPHK, the other one is for another extracellular domain of the same or different SPHK.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, (1983) Nature 305:537-9). Because of the random assortment of immunoglobulin heavy and light chains, these hybridomas (quadromas) produce a potential mixture of ten different antibody molecules, of which only one has the correct bispecific structure. Affinity chromatography steps usually accomplish the purification of the correct molecule. Similar procedures are disclosed in Trauneeker, et al. (1991) EMBO J. 10:3655-9.

According to another preferred embodiment, the assay method comprise using a drug candidate compound identified as having a binding affinity for SPHKs, and/or has already been identified as having down-regulating activity such as antagonist activity vis-à-vis one or more SPHK.

Methods to isolate compounds, and resulting compounds, that inhibit the activity of sphingosine kinases are described by French et al. (Cancer res. 63:5932-5969, 2003). This method is an adaptation of the method of Louie et al (J. Biol. Chem 251:4557-4564, 1976) and uses radio-labeled sphingosine as substrate. Another method involves an ATP competition assay, which is a modification of Olivera et al. (Methods Enzymol., 311,215-223, 2000). These authors identified several compounds that inhibit SPHK1-2 activity. Furthermore natural compounds that inhibit SPHK1-2 have been identified by Kono et al. (J. antibiot. (Tokyo) 54:415-420, 2001), Kono et al. (J. antibiot. 53:753-758, 2000) and Kono et al. (J. Antibiot. (Tokyo) 53:759-764, 2000). The following compounds, identified by number or name designation and structural formulae below, are disclosed in these references, which are incorporated by reference,
A: Compounds 306301-68-8, 312636-16-1, 359899-55-1 and 24388-08-7 (French et al., 2003)
B: DMS (N-dimethylsphingosine, D-erythro (BIOMOL)).
C: S15183A (3. 7-octanoyloxy-3-heptyl-7-methyl-6,8-dioxo-2-oxa-2,6,7,8-tetrahydronaphthalene)
D: F-12509 (C21H2804).

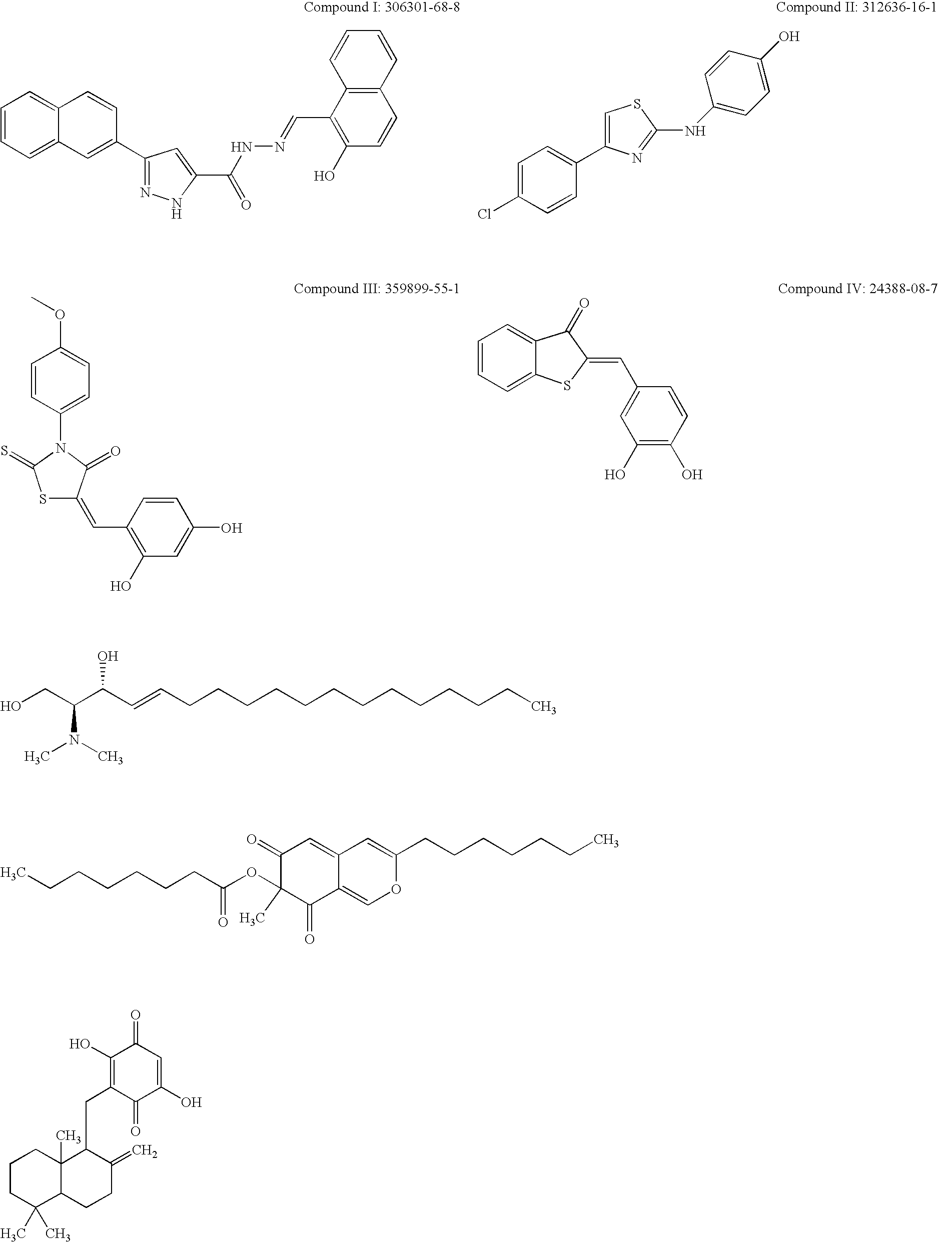

Particularly useful compounds having antagonist activity relative to SPHKs are the following isoflav-3-ene and isoflavan compounds such as Dehydroequol (4′, 7-dihydroxyisoflav-3-ene) and derivatives thereof have been identified as inhibitors of SPHK1-2 (WO 03/086386). Another known inhibitor of SPHKs is D-erythro-N,N-dimethylsphingosine”, aka DMS, Min, J. et al. “Overexpression of Spingosine-1-Phosphate Lyase or Inhibition of Sphingosine Kinase in Dictyostelium disoideum Results in a Selective Increase to Sensitivity to Platinum-based Chemotherapy Drugs” Eukaryotic Cell, 3(3):795-805.

Another aspect of the present invention relates to a method for reducing amyloid-beta precursor protein processing in a mammalian cell, comprising by contacting said cell with an expression-inhibiting agent that inhibits the translation in the cell of a polyribonucleotide encoding a SPHK polypeptide. A particular embodiment relates to a composition comprising a polynucleotide including at least one antisense strand that functions to pair the agent with the target SPHK mRNA, and thereby down-regulate or block the expression of SPHK polypeptide. The inhibitory agent preferably comprises antisense polynucleotide, a ribozyme, and a small interfering RNA (siRNA), wherein said agent comprises a nucleic acid sequence complementary to, or engineered from, a polynucleotide sequence encoding a polypeptide comprising an amino acid sequence selected from the group consisting of SEQ ID NO: 3-4.

A special embodiment of the present invention relates to a method wherein the expression-inhibiting agent is selected from the group consisting of antisense RNA, antisense oligodeoxynucleotide (ODN), a ribozyme that cleaves the polyribonucleotide coding for SEQ ID NO: 3-4, a small interfering RNA (siRNA) that is sufficiently homologous to a portion of the polyribonucleotide corresponding to SEQ ID NO: 3-4 such that the siRNA interferes with the translation of the SPHK polyribonucleotide to the SPHK polypeptide.

Another embodiment of the present invention relates to a method wherein the expression-inhibiting agent is a nucleic acid expressing the antisense RNA, antisense oligodeoxynucleotide (ODN), a ribozyme that cleaves the polyribonucleotide coding for SEQ ID NO: 3-4, a small interfering RNA (siRNA) that is sufficiently homologous to a portion of the polyribonucleotide corresponding to SEQ ID NO: 3-4 such that the siRNA interferes with the translation of the SPHK polyribonucleotide to the SPHK polypeptide. Preferably the expression-inhibiting agent is an antisense RNA, ribozyme, antisense oligodeoxynucleotide, or siRNA comprising a nucleotide sequence selected from the group consisting of SEQ ID NO: 5-34.

The down regulation of gene expression using antisense nucleic acids can be achieved at the translational or transcriptional level. Antisense nucleic acids of the invention are preferably nucleic acid fragments capable of specifically hybridizing with all or part of a nucleic acid encoding a SPHK polypeptide or the corresponding messenger RNA. In addition, antisense nucleic acids may be designed which decrease expression of the nucleic acid sequence capable of encoding a SPHK polypeptide by inhibiting splicing of its primary transcript. Any length of antisense sequence is suitable for practice of the invention so long as it is capable of down-regulating or blocking expression of a nucleic acid coding for a SPHK. Preferably, the antisense sequence is at least about 17 nucleotides in length. The preparation and use of antisense nucleic acids, DNA encoding antisense RNAs and the use of oligo and genetic antisense is known in the art.

One embodiment of expression-inhibitory agent is a nucleic acid that is antisense to a nucleic acid comprising SEQ ID NO: 1-2. For example, an antisense nucleic acid (e.g. DNA) may be introduced into cells in vitro, or administered to a subject in vivo, as gene therapy to inhibit cellular expression of nucleic acids comprising SEQ ID NO: 1-2. Antisense oligonucleotides preferably comprise a sequence containing from about 17 to about 100 nucleotides and more preferably the antisense oligonucleotides comprise from about 18 to about 30 nucleotides. Antisense nucleic acids may be prepared from about 10 to about 30 contiguous nucleotides selected from the sequences of SEQ ID NO: 1-2, expressed in the opposite orientation.

The antisense nucleic acids are preferably oligonucleotides and may consist entirely of deoxyribo-nucleotides, modified deoxyribonucleotides, or some combination of both. The antisense nucleic acids can be synthetic oligonucleotides. The oligonucleotides may be chemically modified, if desired, to improve stability and/or selectivity. Since oligonucleotides are susceptible to degradation by intracellular nucleases, the modifications can include, for example, the use of a sulfur group to replace the free oxygen of the phosphodiester bond. This modification is called a phosphorothioate linkage. Phosphorothioate antisense oligonucleotides are water soluble, polyanionic, and resistant to endogenous nucleases. In addition, when a phosphorothioate antisense oligonucleotide hybridizes to its target site, the RNA-DNA duplex activates the endogenous enzyme ribonuclease (RNase) H, which cleaves the mRNA component of the hybrid molecule.

In addition, antisense oligonucleotides with phosphoramidite and polyamide (peptide) linkages can be synthesized. These molecules should be very resistant to nuclease degradation. Furthermore, chemical groups can be added to the 2′ carbon of the sugar moiety and the 5 carbon (C-5) of pyrimidines to enhance stability and facilitate the binding of the antisense oligonucleotide to its target site. Modifications may include 2′-deoxy, O-pentoxy, O-propoxy, O-methoxy, fluoro, methoxyethoxy phosphorothioates, modified bases, as well as other modifications known to those of skill in the art.

Another type of expression-inhibitory agent that reduces the levels of SPHKs is ribozymes. Ribozymes are catalytic RNA molecules (RNA enzymes) that have separate catalytic and substrate binding domains. The substrate binding sequence combines by nucleotide complementarity and, possibly, non-hydrogen bond interactions with its target sequence. The catalytic portion cleaves the target RNA at a specific site. The substrate domain of a ribozyme can be engineered to direct it to a specified mRNA sequence. The ribozyme recognizes and then binds a target mRNA through complementary base pairing. Once it is bound to the correct target site, the ribozyme acts enzymatically to cut the target mRNA. Cleavage of the mRNA by a ribozyme destroys its ability to direct synthesis of the corresponding polypeptide. Once the ribozyme has cleaved its target sequence, it is released and can repeatedly bind and cleave at other mRNAs.

Ribozyme forms include a hammerhead motif, a hairpin motif, a hepatitis delta virus, group I intron or RNaseP RNA (in association with an RNA guide sequence) motif or Neurospora VS RNA motif. Ribozymes possessing a hammerhead or hairpin structure are readily prepared since these catalytic RNA molecules can be expressed within cells from eukaryotic promoters (Chen, et al. (1992) Nucleic Acids Res. 20:4581-9). A ribozyme of the present invention can be expressed in eukaryotic cells from the appropriate DNA vector. If desired, the activity of the ribozyme may be augmented by its release from the primary transcript by a second ribozyme (Ventura, et al. (1993) Nucleic Acids Res. 21:3249-55).

Ribozymes may be chemically synthesized by combining an oligodeoxyribonucleotide with a ribozyme catalytic domain (20 nucleotides) flanked by sequences that hybridize to the target mRNA after transcription. The oligodeoxyribonucleotide is amplified by using the substrate binding sequences as primers. The amplification product is cloned into a eukaryotic expression vector.

Ribozymes are expressed from transcription units inserted into DNA, RNA, or viral vectors. Transcription of the ribozyme sequences are driven from a promoter for eukaryotic RNA polymerase I (pol (I), RNA polymerase II (pol II), or RNA polymerase III (pol III). Transcripts from pol II or pol III promoters will be expressed at high levels in all cells; the levels of a given pol II promoter in a given cell type will depend on nearby gene regulatory sequences. Prokaryotic RNA polymerase promoters are also used, providing that the prokaryotic RNA polymerase enzyme is expressed in the appropriate cells (Gao and Huang, (1993) Nucleic Acids Res. 21:2867-72). It has been demonstrated that ribozymes expressed from these promoters can function in mammalian cells (Kashani-Sabet, et al. (1992) Antisense Res. Dev. 2:3-15).

A particularly preferred inhibitory agent is a small interfering RNA (siRNA). SiRNAs mediate the post-transcriptional process of gene silencing by double stranded RNA (dsRNA) that is homologous in sequence to the silenced RNA. SiRNA according to the present invention comprises a sense strand of 17-25 nucleotides complementary or homologous to a contiguous 17-25 nucleotide sequence selected from the group of sequences described in SEQ ID NO: 1-2 and an antisense strand of 17-23 nucleotides complementary to the sense strand. The most preferred siRNA comprises sense and anti-sense strands that are 100 percent complementary to each other and the target polynucleotide sequence. Preferably the siRNA further comprises a loop region linking the sense and the antisense strand.

A self-complementing single stranded siRNA molecule polynucleotide according to the present invention comprises a sense portion and an antisense portion connected by a loop region linker. Preferably, the loop region sequence is 4-30 nucleotides long, more preferably 5-15 nucleotides long and most preferably 8 nucleotides long. In a most preferred embodiment the linker sequence is UUGCUAUA (SEQ ID NO: 35). Self-complementary single stranded siRNAs form hairpin loops and are more stable than ordinary dsRNA. In addition, they are more easily produced from vectors.

Analogous to antisense RNA, the siRNA can be modified to confirm resistance to nucleolytic degradation, or to enhance activity, or to enhance cellular distribution, or to enhance cellular uptake, such modifications may consist of modified internucleoside linkages, modified nucleic acid bases, modified sugars and/or chemical linkage the SiRNA to one or more moieties or conjugates. The nucleotide sequences are selected according to siRNA designing rules that give an improved reduction of the target sequences compared to nucleotide sequences that do not comply with these siRNA designing rules (For a discussion of these rules and examples of the preparation of siRNA, WO2004094636, published Nov. 4, 2004, and UA20030198627, are hereby incorporated by reference.

The present invention also relates to compositions, and methods using said compositions, comprising a DNA expression vector capable of expressing a polynucleotide capable of inhibiting amyloid beta protein precursor processing and described hereinabove as an expression inhibition agent.

A special aspect of these compositions and methods relates to the down-regulation or blocking of the expression of a SPHK polypeptide by the induced expression of a polynucleotide encoding an intracellular binding protein that is capable of selectively interacting with the SPHK polypeptide. An intracellular binding protein includes any protein capable of selectively interacting, or binding, with the polypeptide in the cell in which it is expressed and neutralizing the function of the polypeptide. Preferably, the intracellular binding protein is a neutralizing antibody or a fragment of a neutralizing antibody having binding affinity to an intra-cellular domain of the SPHK polypeptide of SEQ ID NO: 3-4. More preferably, the intracellular binding protein is a single chain antibody.

A special embodiment of this composition comprises the expression-inhibiting agent selected from the group consisting of antisense RNA, antisense oligodeoxynucleotide (ODN), a ribozyme that cleaves the polyribonucleotide coding for SEQ ID NO: 3-4, and a small interfering RNA (siRNA) that is sufficiently homologous to a portion of the polyribonucleotide corresponding to SEQ ID NO: 3-4 such that the siRNA interferes with the translation of the SPHK polyribonucleotide to the SPHK polypeptide,

The polynucleotide expressing the expression-inhibiting agent is preferably included within a vector. The polynucleic acid is operably linked to signals enabling expression of the nucleic acid sequence and is introduced into a cell utilizing, preferably, recombinant vector constructs, which will express the antisense nucleic acid once the vector is introduced into the cell. A variety of viral-based systems are available, including adenoviral, retroviral, adeno-associated viral, lentiviral, herpes simplex viral or a sendaviral vector systems, and all may be used to introduce and express polynucleotide sequence for the expression-inhibiting agents in target cells.

Preferably, the viral vectors used in the methods of the present invention are replication defective. Such replication defective vectors will usually pack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention. Retroviral systems and herpes virus system may be preferred vehicles for transfection of neuronal cells.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda P.sub.r, P.sub.1, and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Felgner, et. al. (1987) Proc. Natl. Acad. Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

The present invention also provides biologically compatible compositions comprising the compounds identified as SPHK inhibitors, and the expression-inhibiting agents as described hereinabove.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the SPHK; a nucleic acid would be able to replicate, translate a message, or hybridize to a complementary mRNA of a SPHK; a vector would be able to transfect a target cell and expression the antisense, antibody, ribozyme or siRNA as described hereinabove; an antibody would bind a SPHK polypeptide domain.

A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well-known non-toxic physiologically acceptable carriers, adjuvants and vehicles as desired.

A particularly preferred embodiment of the present composition invention is a cognitive-enhancing pharmaceutical composition comprising a therapeutically effective amount of an expression-inhibiting agent as described hereinabove, in admixture with a pharmaceutically acceptable carrier. Another preferred embodiment is a pharmaceutical composition for the treatment or prevention of a condition involving cognitive impairment or a susceptibility to the condition, comprising an effective amyloid beta peptide inhibiting amount of a SPHK antagonist or inverse agonist its pharmaceutically acceptable salts, hydrates, solvates, or prodrugs thereof in admixture with a pharmaceutically acceptable carrier. A particularly preferred class of such compositions comprise an aryloxydithiourea compound.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the polynucleotide inhibitory agent of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active expression-inhibiting agents may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37.degree. C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The present invention also provides methods of inhibiting the processing of amyloid-beta precursor protein in a subject suffering or susceptible to the abnormal processing of said protein, which comprise the administration to said subject a therapeutically effective amount of an expression-inhibiting agent of the invention. Another aspect of the present method invention is the treatment or prevention of a condition involving cognitive impairment or a susceptibility to the condition. A special embodiment of this invention is a method wherein the condition is Alzheimer's disease.

As defined above, therapeutically effective dose means that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

Antibodies according to the invention may be delivered as a bolus only, infused over time or both administered as a bolus and infused over time. Those skilled in the art may employ different formulations for polynucleotides than for proteins. Similarly, delivery of polynucleotides or polypeptides will be specific to particular cells, conditions, locations, etc.

As discussed hereinabove, recombinant viruses may be used to introduce DNA encoding polynucleotide agents useful in the present invention. Recombinant viruses according to the invention are generally formulated and administered in the form of doses of between about 10.sup.4 and about 10.sup.14 pfu. In the case of AAVs and adenoviruses, doses of from about 10.sup.6 to about 10.sup.11 pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

Still another aspect or the invention relates to a method for diagnosing a pathological condition involving cognitive impairment or a susceptibility to the condition in a subject, comprising determining the amount of polypeptide comprising an amino acid sequence selected from the group consisting of SEQ ID NO: 3-4 in a biological sample, and comparing the amount with the amount of the polypeptide in a healthy subject, wherein an increase of the amount of polypeptide compared to the healthy subject is indicative of the presence of the pathological condition.

### EXPERIMENTAL SECTION

### Example 1

### SPHK1 and SPHK2 Increases Amyloid Beta 1-42 Levels

To identify novel drug targets that change the APP processing, a stable cell line over expressing APP is generated. This stable cell line is made by transfecting HEK293 cells with APP770 wt cDNA cloned into pcDNA3.1, followed by selection with G418 for 3 weeks. At this time point colonies are picked and stable clones are expanded and tested for their secreted amyloid-beta peptide levels. One clone that secretes amyloid-beta at a high level, HEK293 APPwt, is selected for experiments to identify drug targets. This is accomplished by transducing HEK293 APPwt with adenoviral cDNA libraries and measuring changes to the resulting amyloid beta 1-42 levels via ELISA.

Cells seeded in collagen-coated plates at a cell density of 15000 cells/well (384 well plate) in DMEM (10% FBS), are infected 24 h later with 1 μl or 0.2 μl of adenovirus (corresponding to an average multiplicity of infection (MOI) of 120 and 24 respectively). The following day, the virus is washed away and DMEM (25 mM Hepes; 110% FBS) is added to the cells. Amyloid-beta peptides are allowed to accumulate during 24 h. The ELISA plate is prepared by coating with a capture antibody (JRF/cAbeta42/26) (obtained from M Mercken, Johnson and Johnson Pharmaceutical Research and Development, B-2340 Beerse, Belgium) overnight in buffer 42 (Table 2) at a concentration of 2.5 μg/ml. The excess capture antibody is washed away the next morning with PBS and the ELISA plate is then blocked overnight with casein buffer (see Table 2) at 4° C. Upon removal of the blocking buffer, 30 μl of the sample is transferred to the ELISA plate and incubated overnight at 4° C. After extensive washing with PBS-Tween20 and PBS, 30 μl of the horseradish peroxidase (HRP) labeled detection antibody (Peroxidase Labeling Kit, Roche), JRF/AbetaN/25-HRP (obtained from M Mercken, Johnson and Johnson Pharmaceutical Research and Development, B-2340 Beerse, Belgium) is diluted 1/5000 in buffer C (see Table 1) and added to the wells for another 2 h. Following the removal of excess detection antibody by a wash with PBS-Tween20 and PBS, HRP activity is detected via addition of luminol substrate (Roche), which is converted into a chemiluminescent signal by the HRP enzyme.

TABLE 2
buffers and solutions used for ELISA

| | |
|---|---|
| Buffer 42 | 30 mM NaHCO₃, 70 mM Na₂CO₃, 0.05% |
| | NaN₃, pH 9.6 |
| Casein buffer | 0.1% casein in PBS 1× |
| EC Buffer | 20 mM sodium phosphate, 2 mM EDTA, |
| | 400 mM NaCl, 0.2% BSA, 0.05% CHAPS, |
| | 0.4% casein, 0.05% NaN₃, pH 7 |
| Buffer C | 20 mM sodium phosphate, 2 mM EDTA, |
| | 400 mM NaCl, 1% BSA, pH 7 |
| PBS 10× | 80 g NaCl + 2 g KCl + 11.5 g |
| | Na₂HPO₄ · 7H₂O + 2 g KH₂PO₄ |
| | in 11 milli Q, pH 7.4 |
| PBST | PBS 1× with 0.05% Tween 20 |

In order to validate the assay, the effect of adenoviral over expression with random titer of two clinical PS1 mutants and BACE on amyloid beta 1-42 production is evaluated in the HEK293 APPwt cells. As is shown in FIG. 2, all PS1 and BACE constructs induce amyloid beta 1-42 levels as expected.

An adenoviral cDNA library was constructed as follows. DNA fragments are amplified by PCR from a pooled placental and fetal liver cDNA library (InvitroGen). All fragments are cloned into an adenoviral vector as described in U.S. Pat. No. 6,340,595, the contents of which are herein incorporated by reference, and subsequently adenoviruses are made harboring the corresponding cDNAs. During the screening of the adenoviral library in the HEK293 APPwt cells, over expression of SPHK2 lead to increased levels of amyloid beta 1-42 peptides in the conditioned medium of HEK293 APPwt cells. These results indicate that SPHK2 was identified as a modulator of APP processing.

The stimulatory effect of SPHK2 is confirmed upon re-screening of the viruses with a known titer (viral particles/ml), as determined by quantitative real time PCR. SPHK1 and SPHK2 virus is infected at MOIs ranging from 2 to 1250 and the experiment is performed as described above. In addition, the effect of SPHK2 on amyloid beta 1-40, 11-42 and 1-x levels are checked under similar conditions as above. The respective ELISAs are performed as described above, except that the following antibodies were used: for the amyloid beta 1-40 ELISA, the capture and detection antibody are respectively JRF/cAbeta40/10 and JRF/AbetaN/25-HRP (obtained from M Mercken, Johnson and Johnson Pharmaceutical Research and Development, B-2340 Beerse, Belgium), for the amyloid beta 11-42 ELISA, the capture and detection antibody are respectively JRF/cAbeta42/26 and JRF/hAb11/1 (obtained from M Mercken, Johnson and Johnson Pharmaceutical Research and Development, B-2340 Beerse, Belgium), while for the amyloid beta 1-x ELISA (x ranges from 24-42) the capture and detection antibodies are JRF/AbetaN/25 and 4G8-HRP, respectively (obtained respectively from M Mercken, Johnson and Johnson Pharmaceutical Research and Development, B-2340 Beerse, Belgium and from Signet, USA). The amyloid beta 1-x ELISA is used for the detection of amyloid peptides with a variable C-terminus (amyloid beta 1-37; 1-38; 1-39; 1-40; 1-42). The results of these experiments clearly show an increase of amyloid beta 1-40, 11-42, y-42 and 1-x species upon transduction of SPHK. (Figure XX).

### Example 2

### Expression of SPHK2 in Human Brain Tissue

Upon identification of protein kinase involved of APP processing, it is essential to evaluate whether the kinase is expressed in the tissue and cells of interest. This can be achieved by measuring RNA and/or protein levels. In recent years, RNA levels are being quantified through real time PCR technologies, whereby the RNA is first transcribed to cDNA and then the amplification of the cDNA of interest is monitored during a PCR reaction. The amplification plot and the resulting Ct value are indicators for the amount of RNA present in the sample. To assess whether SPHK cDNA is expressed in the human brain, real time PCR with GAPDH specific primers and specific primers for polynucleotides coding for the SPHK polypeptide (Table 3) is performed on human total brain, human cerebral cortex, and human hippocampal total RNA (BD Biosciences). GAPDH RNA is detected with a Taqman probe, while for the polynucleotides of the invention SybrGreen is used. 40 ng of RNA is transcribed to DNA using the MultiScribe Reverse Transcriptase (50 U/μl) enzyme (Applied BioSystems). The resulting cDNA is amplified with AmpliTaq Gold DNA polymerase (Applied BioSystems) during 40 cycles using an ABI PRISM® 7000 Sequence Detection System.

TABLE 3

Primers used in the quantitative real time PCR
analysis for expression levels of the
SPHK polypeptides

| | | | |
|---|---|---|---|
| gene | species | primer name | sequence |

| | | | |
|---|---|---|---|
| SPHK2 | Homo | SPHK2_Hs_For | GCCCCGGTTGCTTCTATTG |
| | Sapiens | | |
| | | SPHK2_Hs_Rev | GTTCTGTCTGGATGAGGTTGAA |
| | Homo | | GG |
| | Sapiens | | |

Total RNA isolated from rat primary neurons and human total brain, cerebral cortex and hippocampal is analyzed, via quantitative real time PCR, for the presence of SPHK cDNA. The Ct values for SPHK2 indicate that SPHK cDNA is detected in all RNA samples (Table 4).

TABLE 4
Ct values obtained during quantitative real time PCR:
Total human brain, human cerebral cortex or human hippocampus
RNA is tested via quantitative real time PCR for the
presence of the respective SPHK RNA

Ct

| Gene | Tissue | RT+ | RT− |
|---|---|---|---|
| SPHK2 | Human Brain Total | 23.90 | 35.26 |
| | RNA | | |
| | Human Brain | 23.59 | 35.05 |
| | Hippocampus Total | | |
| | RNA | | |
| | Human Brain | 23.70 | 33.83 |
| | Cerebral Cortex | | |
| | Total RNA | | |

To gain more insight into the specific cellular expression, immuno-histochemistry (protein level) and/or in situ hybridization (RNA level) is carried out on sections from normal and Alzheimer's human brain hippocampal, cortical and subcortical structures, in diseased and normal tissues. These studies measure expression in neurons, microglia cells and astrocytes, and are able to detect differential SPHK expression between diseased and healthy tissues.

### Example 3

### Reduction of Amyloid Beta Peptide Levels in Neuronal Cells

Human, mouse or rat primary hippocampal or cortical neurons are transduced with adenoviruses expressing the SPHK polypeptides. Amyloid beta levels are determined by ELISA and mass spectrometry analysis (see EXAMPLE 5). Since rodent APP genes carry a number of mutations in APP compared to the human sequence, a detection antibody recognizing rodent amyloid beta is used (JRF/rAb/2; obtained from M Mercken, Johnson and Johnson Pharmaceutical Research and Development, B-2340 Beerse, Belgium). Alternatively, the human amyloid beta ELISAs (see EXAMPLE 1) is performed on cells co-transduction with human wild type APP or human Swedish mutant APP (which enhances amyloid-beta production) cDNA.

Human primary neurons are purchased from Cellial Technologies, France. Rat primary neuron cultures are prepared from brain of E18-E19-day-old fetal Sprague Dawley rats and mouse primary neuron cultures from E14 (cortical cultures) or E17 (cortical and hippocampal cultures)-day old fetal FVB mice, according to Goslin and Banker (Culturing Nerve cells, second edition, 1998 ISBN 0-262-02438-1). Single cell suspensions are prepared from hippocampus or cortical samples. The number of cells is determined (only taking into account the living cells) and cells are plated on poly-L-lysine-coated plastic 96-well plates in minimal essential medium (MEM) supplemented with 10% horse serum. The cells are seeded at a density between 30,000 and 60,000 cells per well (i.e. about 100,000-200,000 cells/cm², respectively). After 3-4 h, culture medium is replaced by 150 μl serum-free neurobasal medium with B27 supplement (GIBCO BRL). Cytosine arabinoside (5 μM) is added 24 h after plating to prevent non-neuronal (glial) cell proliferation.

Neurons are used at day 5-7 after plating. Before adenoviral transduction, 150 μl conditioned medium of these cultures is transferred to the corresponding wells in an empty 96-well plate and 50 μl of the conditioned medium is returned to the cells. The remaining 100 μl/well is stored at 37° C. and 5% CO₂. Both hippocampal and cortical primary neuron cultures are co-infected with the crude lysate of virus containing the cDNAs of the SPHK polypeptides, and human wild type APP or human Swedish mutant APP, at different MOls, ranging from 100 to 3000. Sixteen to twenty-four hours after transduction, virus is removed and cultures are washed with 100 μl pre-warmed fresh neurobasal medium. After removal of the wash solution, the remaining 100 μl of the stored conditioned medium is transferred to the corresponding cells. From this point on, cells secrete amyloid beta peptide into the conditioned medium and its concentration is determined by either rodent or human amyloid beta 1-42 specific ELISAs (see EXAMPLE 1). The conditioned media are collected 24, 48 and 96 hours after exchanging virus-containing medium by stored conditioned medium.

### Example 4

### Amyloid Beta Peptide Reduction Via Knock Down of SPHK Expression

The effect of an antagonist can be mimicked through the use of siRNA-based strategies, which result in decreased expression levels of the targeted protein. Adenoviral mediated siRNA or knock down constructs based upon the sequences shown in Table 5, are constructed as described in WO03/020931.

TABLE 5
KD sequences SEQ IDs:

| | | | |
|---|---|---|---|
| | | | SEQ |
| | | | ID |
| Accession | Code | Sequence | NO |

| | | | |
|---|---|---|---|
| NM_020126_idx555 | SPHK2 | ACGCTTTGCCCTCACCCTTAC | 5 |
| NM_020126idx707 | SPHK2 | ACTTCTGCATCTACACCTACC | 6 |
| NM_020126_idx790 | SPHK2 | ACCTACGAAGAGAACCGTGCC | 7 |
| NM_020126_idx970 | SPHK2 | AACCACGTGCTTCCCATGATC | 8 |
| NM_020126_idx995 | SPHK2 | AAGCTGGGCTGTCCTTCAACC | 9 |
| NM_020126_idx1013 | SPHK2 | ACCTCATCCAGACAGAACGAC | 10 |
| NM_020126_idx1024 | SPHK2 | ACAGAACGACAGAACCACGCC | 11 |
| NM_020126_idx1168 | SPHK2 | AAGATGCCTGTGGGCATCCTC | 12 |
| NM_020126_idx1262 | SPHK2 | ACCTGTTGCTCAACTGCTCAC | 13 |
| NM_020126_idx1273 | SPHK2 | AACTGCTCACTGTTGCTGTGC | 14 |
| NM_020126_idx1274 | SPHK2 | ACTGCTCACTGTTGCTGTGCC | 15 |
| NM_020126_idx1558 | SPHK2 | AAGTCGGAGCTGACCCTAACC | 16 |
| NM_020126_idx1769 | SPHK2 | ACCCACTGCTGTCTTCACCTC | 17 |
| NM_020126_idx1801 | SPHK2 | AAGGCAGCTCTACACTCACCC | 18 |
| NM_020126_idx2332 | SPHK2 | AACTAAACAAGCTTGGTACCC | 19 |
| NM_020126_idx2512 | SPHK2 | AAAGAGAAATGGGCTCGTCCC | 20 |
| NM_020126_idx2755 | SPHK2 | ACTCCGGTGCCTCCATTTAGC | 21 |
| NM_020126_idx2940 | SPHK2 | AAGGCAGTCGCTTCATTCCTC | 22 |
| NM_021972_idx559 | SPHK1 | AAATCTCCTTCACGCTGATGC | 23 |
| NM_021972_idx784 | SPHK1 | ACCATTATGCTGGCTATGAGC | 24 |
| NM_021972_idx810 | SPHK1 | ACCAATGAAGACCTCCTGACC | 25 |
| NM_021972_idx813 | SPHK1 | AATGAAGACCTCCTGACCAAC | 26 |
| NM_021972_idx831 | SPHK1 | AACTGCACGCTATTGCTGTGC | 27 |
| NM_021972_idx832 | SPHK1 | ACTGCACGCTATTGCTGTGCC | 28 |
| NM_021972_idx955 | SPHK1 | ACCTAGAGAGTGAGAAGTATC | 29 |
| NM_021972_idx1070 | SPHK1 | AAGAGTGGGTTCCAAGACACC | 30 |
| NM_021972_idx1183 | SPHK1 | ACGAGGACTTTGTGCTAGTCC | 31 |
| NM_021972_idx1189 | SPHK1 | ACTTTGTGCTAGTCCTGGCAC | 32 |
| NM_021972_idx1491 | SPHK1 | AACTACTTCTGGATGGTCAGC | 33 |
| NM_021972_{—} | SPHK1 | AATAAAGTGACATTCCCAGCC | 34 |
| Loop sequence | | UUGCUAUA | 35 |

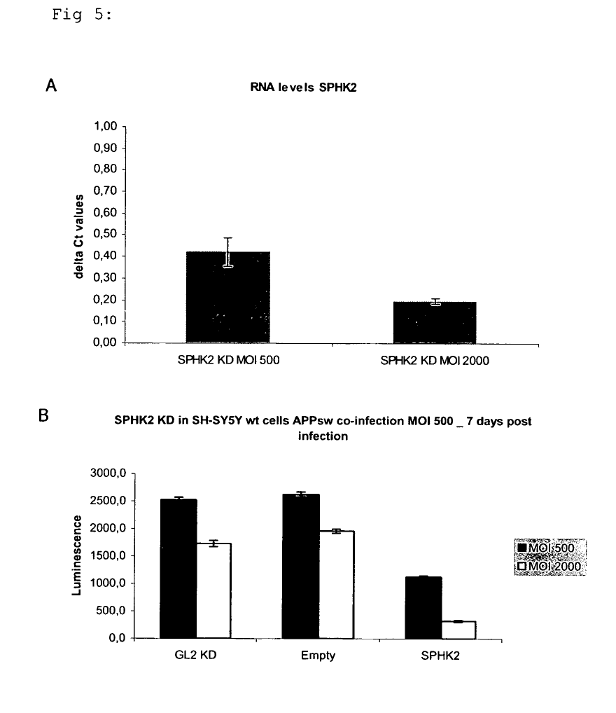

Adenoviral knock down constructs are used to transduce mouse, rat or human primary neuronal cells and/or cell lines (e.g. HEK293, SH-SY5Y, IMR-32, SK-N—SH, SK-N-MC, H4, CHO, COS, HeLa) stably over-expressing APPwt or not. 24 h later, the adenoviruses are removed and fresh medium is added to the cells. 96 h later, the medium of the cells is refreshed to allow the accumulation of amyloid beta 1-42 peptides. After 48 h, the conditioned medium of these cells is assayed using the amyloid beta 1-42 ELISA, which is performed as described in Example 1. Co-infection of SH-SY5Y cells with adenoviruses expressing APPwt and a SPHK2 KD sequence, with nucleotide sequence SEQ ID NO: 6, results in a reduction of amyloid beta 1-42 levels in the conditioned medium compared to GL2 KD virus infected cells (see FIG. 5). In addition, RNA is isolated from these infected cells and SPHK2 RNA levels are determined via real time PCR. Determination of the levels of household keeping genes allows the normalization of RNA levels of the target gene between different RNA samples, represented as delta Ct values. The data show clearly that SPHK2 RNA levels are reduced in cells infected with the SPHK2 adenoviral KD virus, and that SPHK2 modulates the levels of secreted amyloid beta peptide.

### Example 5

### Mass Spec of Amyloid Beta Peptide in SPHK-Transduced Cell Medium

A mass spectrometry analysis is carried out on the immuno-precipitated conditioned medium of cells exhibiting increased or reduced levels of the SPHK polypeptides to determine specifically how APP processing is modulated by the SPHK polypeptides.

24 well plates (Cellstar, Greiner Bio-One) are coated with collagen (5 μg/ml) for 4 h at 37° C. After replacement of the collagen by medium (DMEM from GIBCO with 10% heat-inactivated FBS from ICN), HEK293 APP770 wt cells are seeded at a density of 0.17×10⁶ cells per well. Cells are grown overnight at 37° C., 10% CO₂. Cells are then infected with the crude lysate of expression vector virus containing the SPHK polynucleotide sequences at the appropriate MOIs. The cells are incubated at 37° C., 10% CO₂. After 20 to 24 hours, the cell culture medium is removed by aspiration and 1 ml of fresh medium (DMEM, 0.2% heat-inactivated FBS, 1X ITS from GIBCO) is added to the cells. 24 hours later, the conditioned medium is harvested. Protease inhibitors (Roche) are added immediately and the samples are kept on ice in eppendorf tubes or stored at −80° C. until further processing.

After rigorously vortexing the Protein G Sepharose beads (Amersham Biosciences), 5 μl of the slurry is added to each tube, together with 1 μg of specific antibody e.g. 4G8 or JRF/cAbeta42/26 (obtained from M Mercken, Johnson and Johnson Pharmaceutical Research and Development, B-2340 Beerse, Belgium). Tubes are rotated overnight at 4° C. After aspiration of the supernatant, beads are washed twice by adding 850 μl of wash buffer (10 mM Tris-HCl (pH 8.0) containing 0.1% n-octylglucoside, 150 mM NaCl, 0.025% sodium azide) and centrifuging at 20,800 g for 5 min. After a final wash step with 850 μl of 10 mM Tris-HCl (pH 8.0), beads are centrifuged again and the supernatant is removed completely. The pelleted beads are stored at −80° C. until further analysis.

A saturated solution of matrix (alpha-cyano-4-hydroxy-cinnamic acid, Sigma-Aldrich) is prepared in 50% acetonitrile/0.1% TFA containing 0.1 μg/ml synthetic amyloid beta 12-28 (Sigma-Aldrich). 3.5 μl of this elution buffer is added to the thawed dry beads and sonicated for 30 s in a water bath (Branson 200)) at room temperature. The samples are spun 1 min at 20,800 g.

One μl of eluted sample is directly spotted on a ground stainless steel 384 MALDI target plate (Bruker Daltronics). Samples are allowed to air dry until crystallization of matrix/sample mix. The samples are analyzed using a Ultraflex TOF/TOF mass spectrometer (Bruker Daltronics). The resulting spectra are calibrated using a standard curve acquired using a mixture of several standard peptides in the mass range of 1200-3200 Da (Sigma).

Spectra are analyzed using Xtof version 5.1.5 (Bruker Daltronics) and monoisotopic peak intensities (highest peak is used for Abeta1-42) exported to Excel 2000 for further processing. Data are presented as relative ratios to the standard 12-28 peptide.

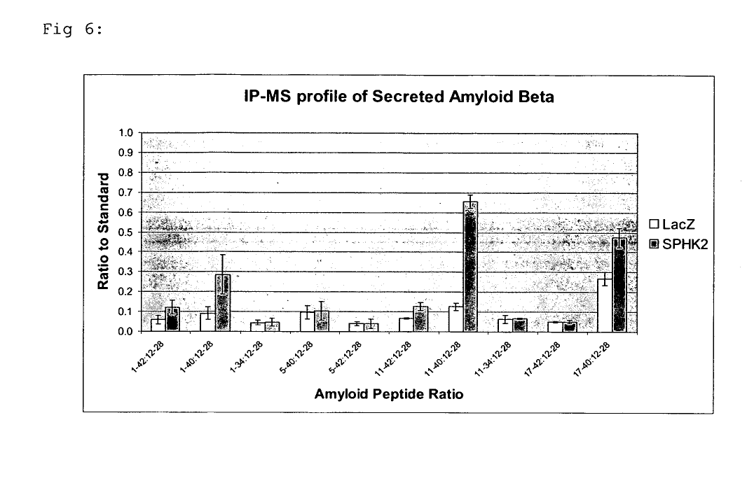

Comparative Measurement: HEK293 APP770 wt cells are infected with adenoviruses containing either a cDNA encoding for LacZ or SPHK2 at MOI 1500, as described above. Quantitative mass spectrometry analysis of immuno-precipitated amyloid peptides from the conditioned medium show a 2-fold increase in amyloid beta peptide 1-42 in media from cells transduced with by SPHK2 compared with LacZ control virus (FIG. 6). Increases in other biologically relevant amyloid peptides were also observed, namely the amyloid beta 1-40, 11-40, 11-42 and 17-40 peptides, confirming the ELISA data.

### Example 6

### Identification of Small Molecules That Inhibit Kinase Activity

Compounds are screened for inhibition of the activity of the SPHK polypeptides. The affinity of the compounds to the polypeptides is determined in an experiment detecting changed reaction conditions after phosphorylation. The SPHK polypeptides are incubated with its substrate and ATP in an appropriate buffer. The combination of these components results in the in vitro phosphorylation of the substrate. Sources of compounds include commercially available screening library, peptides in a phage display library or an antibody fragment library, and compounds that have been demonstrated to have binding affinity for a SPHK, that is a polypeptide comprising an amino acid sequence selected from the group consisting of SEQ ID NO: 3-4.

The SPHK polypeptides can be prepared in a number of ways depending on whether the assay will be run using cells, cell fractions or biochemically, on purified proteins. The polypeptides can be applied as complete polypeptides or as polypeptide fragments, which still comprise SPHK catalytic activity.

Identification of small molecules inhibiting the activity of the SPHK polypeptides is performed by measuring changes in levels of phosphorylated substrate or ATP. Since ATP is consumed during the phosphorylation of the substrate, its levels correlate with the kinase activity. Measuring ATP levels via chemiluminescent reactions therefore represents a method to measure kinase activity in vitro (Perkin Elmer). In a second type of assay, changes in the levels of phosphorylated substrate are detected with phosphospecific agents and are correlated to kinase activity. These levels are detected in solution or after immobilization of the substrate on a microtiter plate or other carrier. In solution, the phosphorylated substrate is detected via fluorescence resonance energy transfer (FRET) between the Eu labeled substrate and an APC labeled phosphospecific antibody (Perkin Elmer), via fluorescence polarization (FP) after binding of a phosphospecific antibody to the fluorescently labeled phosphorylated substrate (Panvera), via an Amplified Luminescent Proximity Homogeneous Assay (ALPHA) using the phosphorylated substrate and phosphospecific antibody, both coupled to ALPHA beads (Perkin Elmer) or using the IMAP binding reagent that specifically detects phosphate groups and thus alleviates the use of the phosphospecific antibody (Molecular Devices). Alternatively, the substrate is immobilized directly or by using biotin-streptavidin on a microtiter plate. After immobilization, the level of phosphorylated substrate is detected using a classical ELISA where binding of the phosphospecific antibody is either monitored via an enzyme such as horseradish peroxidase (HRP) or alkaline phospahtase (AP) which are either directly coupled to the phosphospecific antibody or are coupled to a secondary antibody. Enzymatic activity correlates to phosphorylated substrate levels. Alternatively, binding of the Eu-labeled phosphospecific antibody to the immobilized phosphorylated substrate is determined via time resolved fluorescence energy (TRF) (Perkin Elmer). In addition, the substrate can be coated on FLASH plates (Perkin Elmer) and phosphorylation of the substrate is detected using ³³P labeled ATP or ¹²⁵I, labeled phosphospecific antibody.

Phosphorylated substrate levels are measured directly or indirectly using a tracer. Specifically, if the substrate is a lipid, the kinase activity is detected after extraction of the hydrophilic phosphorylated substrate, which is radioactively labeled. The radioactive signal in the aqueous phase then correlates to the kinase activity (French et al, 2003).

Small molecules are randomly screened or are preselected based upon drug class, (i.e. known kinase inhibitors), or upon virtual ligand screening (VLS) results. VLS uses virtual docking technology to test large numbers of small molecules in silico for their binding to the polypeptide of the invention. Small molecules are added to the kinase reaction and their effect on levels of phosphorylated substrate is measured with one or more of the above-described technologies.

Small molecules that inhibit the kinase activity are identified and are subsequently tested at different concentrations. IC₅₀ values are calculated from these dose response curves. Strong binders have an IC₅₀ in the nanomolar and even picomolar range. Compounds that have an IC₅₀ of at least 10 micromol or better (nmol to pmol) are applied in amyloid beta secretion assay to check for their effect on the beta amyloid secretion and processing.

1. A method for identifying a compound that inhibits the processing of amyloid-beta precursor protein in a mammalian cell, comprising
(a) contacting a compound with a polypeptide comprising an amino acid sequence selected from the group consisting of SEQ ID NO: 3-4; and
(b) measuring a compound-polypeptide property related to the production of amyloid-beta peptide.

2. The method according to claim 1, wherein said polypeptide is in an in vitro cell-free preparation.

3. The method according to claim 2, wherein said polypeptide is present in a mammalian cell.

4. The method of claim 1, wherein said property is a binding affinity of said compound to said polypeptide.

5. The method of claim 3, wherein said property is activation of a biological pathway producing an indicator of the processing of amyloid-beta precursor protein.

6. The method of claim 5 wherein said indicator is a phosphorylated substrate of a kinase.

7. The method of claim 6 wherein said indicator is sphingosine-1-phosphate.

8. The method of claim 5 wherein said indicator is amyloid-beta peptide.

9. The method of claim 8 wherein said amyloid-beta peptide is selected from the group consisting of one or more of amyloid-beta peptide 1-42, 1-40, 11-42 and 11-40.

10. The method of claim 9 wherein said amyloid-beta peptide is amyloid-beta peptide 1-42.

11. The method according to claim 5 wherein said indicator induces the expression of a reporter in said mammalian cell.

12. The method according to claim 11 wherein the reporter is selected from the group consisting of alkaline phosphatase, GFP, eGFP, dGFP, luciferase and B-galactosidase.

13. The method according to claim 1, wherein said compound is selected from the group consisting of compounds of a commercially available screening library and compounds having binding affinity for a polypeptide comprising an amino acid sequence selected from the group consisting of SEQ ID NO: 3-4.

14. The method according to claim 2, wherein said compound is a peptide in a phage display library or an antibody fragment library.

15. The method according to claim 1, wherein said compound is an isoflav-3-ene, an isoflavan, or dehydroequol.

16. An agent for the inhibition of amyloid-beta precursor processing selected from the group consisting of an antisense polynucleotide, a ribozyme, and a small interfering RNA (siRNA), wherein said agent comprises a nucleic acid sequence complementary to, or engineered from, a naturally-occurring polynucleotide sequence encoding a polypeptide comprising an amino acid sequence selected from the group consisting of SEQ ID NO: 3-4.

17. The agent according to claim 16, wherein a vector in a mammalian cell expresses said agent.

18. The agent according to claim 17, wherein said vector is an adenoviral, retroviral, adeno-associated viral, lentiviral, a herpes simplex viral or a sendaiviral vector.

19. The agent according to claim 18, wherein said antisense polynucleotide and said siRNA comprise an antisense strand of 17-25 nucleotides complementary to a sense strand, wherein said sense strand is selected from 17-25 continuous nucleotides of a nucleic acid sequence selected from the group consisting of SEQ ID NO: 5-34 and 36-65.

20. The agent according to claim 19, wherein said siRNA further comprises said sense strand.

21. The agent according to claim 19, wherein said sense strand is selected from 17-25 continuous nucleotides of a nucleic acid sequence selected from the group consisting of SEQ ID NO: 1-2.

22. The agent according to claim 16, wherein said siRNA further comprises a loop region connecting said sense and said antisense strand.

23. The agent according to claim 22 wherein said loop region comprises a nucleic acid sequence defined of SEQ ID NO: 35.

24. The agent according to claim 16, wherein said agent is an antisense polynucleotide, ribozyme, or siRNA comprising a nucleic acid sequence complementary to a sequence selected from the group consisting of SEQ ID NO: 5-34 and 36-65.

25. A cognitive enhancing pharmaceutical composition comprising a therapeutically effective amount of an agent of claim 16 in admixture with a pharmaceutically acceptable carrier.

26. The cognitive enhancing pharmaceutical composition according to claim 25 wherein said agent comprises a polynucleotide comprising a nucleic acid sequence selected from the group consisting of SEQ ID NO: 5-34 and 36-65, a polynucleotide complementary to said nucleic acid sequence, and a combination thereof.

27. A method of inhibiting the processing of amyloid-beta precursor protein in a subject suffering or susceptible to the abnormal processing of said protein, comprising administering to said subject a pharmaceutical composition according to claim 25.

28. A method according to claim 27 for treatment or prevention of a condition involving cognitive impairment or a susceptibility to the condition.

29. The method according to claim 28 wherein the condition is Alzheimer's disease.

30. A pharmaceutical composition for the treatment or prevention of a condition involving cognitive impairment or a susceptibility to the condition, comprising an effective amyloid-beta precursor processing-inhibiting amount of a sphingosine-1-kinase inhibitor.

31. A composition according to claim 30, wherein said sphingosine-1-kinase inhibitor is selected from the group consisting of an isoflav-3-ene, an isoflavan, dehydroequol, and pharmaceutically acceptable salts, hydrates, solvates, or prodrugs thereof in admixture with a pharmaceutically acceptable carrier.


SEQUENCE LISTING (35 sequences)

SEQ ID NO: 1 - DNA, 3012 bp, Homo sapiens
cgactgactagccgggcgataacggcagagagcatagagcgcaggaacaagcgcaacgtccaagagggaagggccagcacgtcgggggcctctctggccctacccaggccgtgttctcgatagctttccggaagaaagggatctgggagcgagatgcgtgtagctagcacgatgcgtcgcgcggtgacgctctggcccgacgccgacggcctctcagtggctcccggaggacccggcgggcccagtgttggagagctgaaggtcaggccaggacagtgagacctgactccttgctcctaccagcctactatggcttaagacccagggccagggtcccgttgatgtaacagagcagaggaccagcagatgaatggacaccttgaagcagaggagcagcaggaccagaggccagaccaggagctgaccgggagctggggccacgggcctaggagcaccctggtcagggctaaggccatggccccgcccccaccgccactggctgccagcaccccgctcctccatggcgagtttggctcctacccagcccgaggcccacgctttgccctcacccttacatcgcaggccctgcacatacagcggctgcgccccaaacctgaagccaggccccggggtggcctggtcccgttggccgaggtctcaggctgctgcaccctgcgaagccgcagcccctcagactcagcggcctacttctgcatctacacctaccctcggggccggcgcggggcccggcgcagagccactcgcaccttccgggcagatggggccgccacctacgaagagaaccgtgccgaggcccagcgctgggccactgccctcacctgtctgctccgaggactgccactgcccggggatggggagatcacccctgacctgctacctcggccgccccggttgcttctattggtcaatccctttgggggtcggggcctggcctggcagtggtgtaagaaccacgtgcttcccatgatctctgaagctgggctgtccttcaacctcatccagacagaacgacagaaccacgcccgggagctggtccaggggctgagcctgagtgagtgggatggcatcgtcacggtctcgggagacgggctgctccatgaggtgctgaacgggctcctagatcgccctgactgggaggaagctgtgaagatgcctgtgggcatcctcccctgcggctcgggcaacgcgctggccggagcagtgaaccagcacgggggatttgagccagccctgggcctcgacctgttgctcaactgctcactgttgctgtgccggggtggtggccacccactggacctgctctccgtgacgctggcctcgggctcccgctgtttctccttcctgtctgtggcctggggcttcgtgtcagatgtggatatccagagcgagcgcttcagggccttgggcagtgcccgcttcacactgggcacggtgctgggcctcgccacactgcacacctaccgcggacgcctctcctacctccccgccactgtggaacctgcctcgcccacccctgcccatagcctgcctcgtgccaagtcggagctgaccctaaccccagacccagccccgcccatggcccactcacccctgcatcgttctgtgtctgacctgcctcttcccctgccccagcctgccctggcctctcctggctcgccagaacccctgcccatcctgtccctcaacggtgggggcccagagctggctggggactggggtggggctggggatgctccgctgtccccggacccactgctgtcttcacctcctggctctcccaaggcagctctacactcacccgtctccgaaggggcccccgtaattcccccatcctctgggctcccacttcccacccctgatgcccgggtaggggcctccacctgcggcccgcccgaccacctgctgcctccgctgggcaccccgctgcccccagactgggtgacgctggagggggactttgtgctcatgttggccatctcgcccagccacctaggcgctgacctggtggcagctccgcatgcgcgcttcgacgacggcctggtgcacctgtgctgggtgcgtagcggcatctcgcgggctgcgctgctgcgccttttcttggccatggagcgtggtagccacttcagcctgggctgtccgcagctgggctacgccgcggcccgtgccttccgcctagagccgctcacaccacgcggcgtgctcacagtggacggggagcaggtggagtatgggccgctacaggcacagatgcaccctggcatcggtacactgctcactgggcctcctggctgcccggggcgggagccctgaaactaaacaagcttggtacccgccgggggcggggcctacattccaatggggcggagcctgagctagggggtgtggcctggctgctagagttgtggtggcaggggccctggccccgtctcaggattgcgctcgctttcatgggaccagacgtgatgctggaaggtgggcgtcgtcacggttaaagagaaatgggctcgtcccgagggtagtgcctgatcaatgagggcggggcctggcgtctgatctggggccgcccttacggggcagggctcagtcctgacgcttgccacctgctcctacccggccaggatggctgagggcggagtctattttacgcgtcgcccaatgacaggacctggaatgtactggctggggtaggcctcagtgagtcggccggtcagggcccgcagcctcgccccatccactccggtgcctccatttagctggccaatcagcccaggaggggcaggttccccggggccggcgctaggatttgcactaatgttcctctccccgcgggtgggggcggggaaattcatatcccctgttcgtctcatgcgcgtcctccgtccccaatctaaaaagcaattgaaaaggtctatgcaataaaggcagtcgcttcattcctctcaaaaaaaaaaaaaaaaaaaaaaaaaaaaaaaaaaaaaaaaaaaaaaaaaa

SEQ ID NO: 2 - DNA, 1869 bp, Homo sapiens
gctccgcggcgccggctgcgaagttgagcgaaaagtttgaggccggagggagcgaggccggggagtccgctccagcggggcgctccagtccctcagacgtgggctgagcttgggacgagctgcgttccgccccaggccactgtagggaacggcggtggcgcctccccagcaaaccggaccgactgggtccagccgccgcagggaatgacaccggtgctcctacagccacggctccgggcggggaaggcgagccccacagccggccctgcgacgccctcctgggcagcaccgataaggagctgaaggcaggagccgccgccacgggcagcgcccccacagcgccagggaccccctggcagcgggagccgcgggtcgaggttatggatccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatgacccctgggccgcgctgtgccttagtgtctacttgcaggacccttcctccttccctagggctgcagagcctgtccacagctcctgtgggggtggaggagactcctctggagaagggtgagaaggtggaggctatgctttggggggacaggccagaatgaagtcctgggtcaggagcccagctggctgggcccagctgcctatgtaaggccttctagtttgttctgagacccccaccccacgaaccaaatccaaataaagtgacattcccagcctgaaaaaaaaaaaaaaaaaa

SEQ ID NO: 3 - protein, 654 aa, Homo sapiens
MNGHLEAEEQQDQRPDQELTGSWGHGPRSTLVRAKAMAPPPPPLAASTPLLHGEFGSYPARGPRFALTLTSQALHIQRLRPKPEARPRGGLVPLAEVSGCCTLRSRSPSDSAAYFCIYTYPRGRRGARRRATRTFRADGAATYEENRAEAQRWATALTCLLRGLPLPGDGEITPDLLPRPPRLLLLVNPFGGRGLAWQWCKNHVLPMISEAGLSFNLIQTERQNHARELVQGLSLSEWDGIVTVSGDGLLHEVLNGLLDRPDWEEAVKMPVGILPCGSGNALAGAVNQHGGFEPALGLDLLLNCSLLLCRGGGHPLDLLSVTLASGSRCFSFLSVAWGFVSDVDIQSERFRALGSARFTLGTVLGLATLHTYRGRLSYLPATVEPASPTPAHSLPRAKSELTLTPDPAPPMAHSPLHRSVSDLPLPLPQPALASPGSPEPLPILSLNGGGPELAGDWGGAGDAPLSPDPLLSSPPGSPKAALHSPVSEGAPVIPPSSGLPLPTPDARVGASTCGPPDHLLPPLGTPLPPDWVTLEGDFVLMLAISPSHLGADLVAAPHARFDDGLVHLCWVRSGISRAALLRLFLAMERGSHFSLGCPQLGYAAARAFRLEPLTPRGVLTVDGEQVEYGPLQAQMHPGIGTLLTGPPGCPGREP

SEQ ID NO: 4 - protein, 398 aa, Homo sapiens
MDPVVGCGRGLFGFVFSAGGPRGVLPRPCRVLVLLNPRGGKGKALQLFRSHVQPLLAEAEISFTLMLTERRNHARELVRSEELGRWDALVVMSGDGLMHEVVNGLMERPDWETAIQKPLCSLPAGSGNALAASLNHYAGYEQVTNEDLLTNCTLLLCRRLLSPMNLLSLHTASGLRLFSVLSLAWGFIADVDLESEKYRRLGEMRFTLGTFLRLAALRTYRGRLAYLPVGRVGSKTPASPVVVQQGPVDAHLVPLEEPVPSHWTVVPDEDFVLVLALLHSHLGSEMFAAPMGRCAAGVMHLFYVRAGVSRAMLLRLFLAMEKGRHMEYECPYLVYVPVVAFRLEPKDGKGVFAVDGELMVSEAVQGQVHPNYFWMVSGCVEPPPSWKPQQMPPPEEPL

SEQ ID NO: 5 - DNA, 19 bp, Homo sapiens
gctttgccctcacccttac

SEQ ID NO: 6 - DNA, 19 bp, Homo sapiens
ttctgcatctacacctacc

SEQ ID NO: 7 - DNA, 19 bp, Homo sapiens
ctacgaagagaaccgtgcc

SEQ ID NO: 8 - DNA, 19 bp, Homo sapiens
ccacgtgcttcccatgatc

SEQ ID NO: 9 - DNA, 19 bp, Homo sapiens
gctgggctgtccttcaacc

SEQ ID NO: 10 - DNA, 19 bp, Homo sapiens
ctcatccagacagaacgac

SEQ ID NO: 11 - DNA, 19 bp, Homo sapiens
agaacgacagaaccacgcc

SEQ ID NO: 12 - DNA, 19 bp, Homo sapiens
gatgcctgtgggcatcctc

SEQ ID NO: 13 - DNA, 19 bp, Homo sapiens
ctgttgctcaactgctcac

SEQ ID NO: 14 - DNA, 19 bp, Homo sapiens
ctgctcactgttgctgtgc

SEQ ID NO: 15 - DNA, 19 bp, Homo sapiens
tgctcactgttgctgtgcc

SEQ ID NO: 16 - DNA, 19 bp, Homo sapiens
gtcggagctgaccctaacc

SEQ ID NO: 17 - DNA, 19 bp, Homo sapiens
ccactgctgtcttcacctc

SEQ ID NO: 18 - DNA, 19 bp, Homo sapiens
ggcagctctacactcaccc

SEQ ID NO: 19 - DNA, 19 bp, Homo sapiens
ctaaacaagcttggtaccc

SEQ ID NO: 20 - DNA, 19 bp, Homo sapiens
agagaaatgggctcgtccc

SEQ ID NO: 21 - DNA, 19 bp, Homo sapiens
tccggtgcctccatttagc

SEQ ID NO: 22 - DNA, 19 bp, Homo sapiens
ggcagtcgcttcattcctc

SEQ ID NO: 23 - DNA, 19 bp, Homo sapiens
atctccttcacgctgatgc

SEQ ID NO: 24 - DNA, 19 bp, Homo sapiens
cattatgctggctatgagc

SEQ ID NO: 25 - DNA, 19 bp, Homo sapiens
caatgaagacctcctgacc

SEQ ID NO: 26 - DNA, 19 bp, Homo sapiens
tgaagacctcctgaccaac

SEQ ID NO: 27 - DNA, 19 bp, Homo sapiens
ctgcacgctattgctgtgc

SEQ ID NO: 28 - DNA, 19 bp, Homo sapiens
tgcacgctattgctgtgcc

SEQ ID NO: 29 - DNA, 19 bp, Homo sapiens
ctagagagtgagaagtatc

SEQ ID NO: 30 - DNA, 19 bp, Homo sapiens
gagtgggttccaagacacc

SEQ ID NO: 31 - DNA, 19 bp, Homo sapiens
gaggactttgtgctagtcc

SEQ ID NO: 32 - DNA, 19 bp, Homo sapiens
tttgtgctagtcctggcac

SEQ ID NO: 33 - DNA, 19 bp, Homo sapiens
ctacttctggatggtcagc

SEQ ID NO: 34 - DNA, 19 bp, Homo sapiens
taaagtgacattcccagcc

SEQ ID NO: 35 - RNA, 8 nt, Homo sapiens
uugcuaua